(19) United States
(12) Patent Application Publication
(10) Pub. No.: US 2009/0097881 A1
(43) Pub. Date: Apr. 16, 2009

(54) ELECTROPHOTOGRAPHIC PHOTORECEPTOR CONTAINING TRIAMINE COMPOUND AND IMAGE FORMING APPARATUS HAVING THE SAME, AS WELL AS TRIAMINE COMPOUND AND METHOD FOR PRODUCING THE SAME

(76) Inventors: Akihiro Kondoh, Nara-shi (JP); Takatsugu Obata, Nara-shi (JP)

Correspondence Address: NIXON & VANDERHYE, PC, 901 NORTH GLEBE ROAD, 11TH FLOOR, ARLINGTON, VA, 22203, US

(21) Appl. No.: 12/251,504

(22) Filed: Oct. 15, 2008

(30) Foreign Application Priority Data
Oct. 16, 2007 (JP) 2007-269256

(51) Int. Cl. G03G 15/02; C07D 251/04

(52) U.S. Cl. 399/174; 430/58.5; 544/180

(57) ABSTRACT

To provide a novel triamine compound usable for providing an electrophotographic photoreceptor having excellent in effect of an ozone resistance, and causing no adverse effect in other characteristic aspects, and a method for producing the same, as well as an electrophotographic photoreceptor using the triamine compound, and an image forming apparatus having the same, there is provided the electrophotographic photoreceptor formed by laminating a monolayer type photosensitive layer containing a charge generating material and a charge transporting material, or a laminate type photosensitive layer having a charge generating layer containing the charge generating material and a charge transporting layer containing the charge transporting material laminated in this order, on a conductive supporting member made of a conductive material, the monolayer type photosensitive layer or the charge transporting layer of the laminate type photosensitive layer containing a specific triamine compound.

### CROSS-REFERENCE TO RELATED APPLICATION

This application is related to Japanese Patent Application No. 2007-269256 filed on 16 Oct. 2008. whose priority is claimed under 35 USC § 119, the disclosure of which is incorporated by reference in its entirety.

### BACKGROUND OF THE INVENTION

1. Field of the Invention

The present invention relates to an electrophotographic photoreceptor containing a triamine compound, and an image forming apparatus having the same, as well as the triamine compound and a method for producing the same.

2. Description of the Related Art

An image forming apparatus of an electrophotographic system (hereinafter, also referred to as an “electrophotographic apparatus”) which forms an image using an electrophotographic technique is often used as a copying machine, a printer, a facsimile machine and the like.

In the electrophotographic apparatus, the image is formed through an electrophotographic process as follows.

First, after charging a photosensitive layer of an electrophotographic photoreceptor (hereinafter, also referred to as a “photoreceptor”) provided in the apparatus, an electrostatic latent image is formed by a light exposure.

The formed electrostatic latent image is developed to form a toner image, and then the formed toner image is transferred and fixed onto a transfer material such as a recording paper, and thus a desired image is formed on the transfer material.

In recent years, the electrophotographic technique is used also in fields of printing plate materials, slide films, microfilms and the like where conventionally a silver halide photographic technique is used, as well as in fields of the copying machines, and is also applied in a high-speed printer which uses laser, LED (light-emitting diode), CRT (cathode ray tube) or the like as an optical source. With such an expansion of application fields of the electrophotographic technique, a demand for a photoreceptor is becoming higher and wider.

As the photoreceptor, conventionally, an inorganic photoreceptor having a photosensitive layer based on an inorganic photoconductive material such as selenium, zinc oxide, cadmium sulfide has been widely used.

Although the inorganic photoreceptor has certain degree of fundamental characteristics of the photoreceptor, it has drawbacks of difficulty in a film formation of the photosensitive layer, a poor plasticity, a high production cost and the like. In addition, since the inorganic photoconductive material has generally strong toxicity, significant limitations arise in production and handling.

As described above, since the inorganic photoconductive material and the inorganic photoreceptor using the same have many drawbacks, a research and development for an organic photoconductive material has proceeded.

The organic photoconductive material has been widely researched and developed in recent years, and not only utilized in an electrostatic recording device such as photoreceptor, but also started to be applied to a sensor device, an organic EL (electro luminescent) device and the like.

An organic photoreceptor using the organic photoconductive material has been developed as a leading photoreceptor because it advantageously has an excellent film formability of the photosensitive layer, an excellent flexibility and allows an easy designing of the photoreceptor, and also has a light weight and an excellent transparency and shows an excellent sensitivity in a wide wavelength range as a result of an appropriate sensitizing method.

Although an early organic photoreceptor had drawbacks in sensitivity and durability, these drawbacks have been significantly improved by a development of a function separated type photoreceptor in which a charge generating function and a charge transporting function are assigned to different substances. This function separated type photoreceptor also has advantages that a material constituting the photosensitive layer can be selected from a wide range and the photoreceptor having an appropriate characteristic can be produced relatively easily, in addition to the advantages realized by the organic photoreceptor as described above.

Various structures have been proposed as a structure of such organic photoreceptor, including such as a monolayer structure wherein both a charge generating material and a charge transporting material (also referred to as a “charge transfer substance”) are dispersed in a binder resin on a supporting member; and a laminate structure or a reverse double-layer type laminate structure wherein a charge generating layer in which the charge generating material is dispersed in the binder resin, and a charge transporting layer in which the charge transporting material is dispersed in the binder resin are formed on the supporting member in this order or in a reverse order.

Among them, a function separated type photoreceptor having the photosensitive layer in which the charge transporting layer is laminated on the charge generating layer is widely brought into practical use because it has an excellent electrophotographic characteristic and durability, and allows various designs of photoreceptor characteristics owing to wide variance of material selection.

As the charge generating material used in the function separated type photoreceptor, various substances including a phthalocyanine pigment, a squarylium pigment, an azo pigment, a perylene pigment, a multicyclic quinone pigment, a cyanine pigment, a squaric acid dye, a pyrylium salt pigment and the like have been discussed, and various materials having a strong light resistance and a high charge generating ability have been proposed.

Further, as the charge transporting material, various compounds including such as a pyrazoline compound, a hydrazone compound, a triphenylamine compound, a stilbene compound and an enamine compound are known.

The photoreceptor having such a proposed or discussed configuration is requested to have various performances including such as a high speed, durability and a stability of sensitivity. In particular, an achievement of both a high sensitivity for corresponding to the high speed, and increased durability, or elongated life time by improvement of an abrasion resistance and the stability of sensitivity is requested as the characteristic of the photoreceptor, in correspondence with a reverse development type electrophotographic apparatus such as a recent digital copying machine and a laser printer. In addition, a higher image reliability and a repetition stability are requested for the photoreceptor used, for example, in the laser printer.

However, it has been argued that one great drawback of such photoreceptor is generally lower durability compared with the inorganic photoreceptor. The durability is roughly classified into durability in aspects of an electrophotographic physical property such as sensitivity, a residual potential, a charging ability and image fuzziness, and a mechanical durability such as an abrasive and a flaw on a surface of the photoreceptor by rubbing. It is known that a reduction in durability in an aspect of the electrophotographic physical property is mainly attributed to deterioration in charge transporting material contained in a surface layer of the photoreceptor, caused by ozone generating by corona discharge, NOₓ (nitrogen oxide) or the like and light irradiation. Although a number of proposed charge transporting materials having various backbones have been significantly improved in an aspect of durability, it is a current state of art that such improvement is not still sufficient.

In addition, the photoreceptor is used repeatedly in a system, and hence a constant stable electrophotographic characteristic is required. At present, such stability and durability have not been sufficiently obtained in any of configurations.

In other words, a decrease in potential, a rise in residual potential, a change in sensitivity and the like occur with a repeated usage, and a copy quality decreases to become no longer usable. Although not all of causes of these deteriorations have been elucidated, some expectable factors are as follows.

For example, it has been proved that an oxidized gas such as ozone discharged from a corona discharge and charge device and nitrogen oxide causes a significant damage on the photosensitive layer. Such oxidized gas causes a chemical change on the material in the photosensitive layer to lead various characteristic changes.

For example, it causes a decrease in charging potential, the rise in residual potential, and a deterioration in resolving power due to a decrease in surface resistance, with a result that the image fuzziness such as a pin hole and a black band occur on an output image to significantly impair the image quality, and a life time of the photoreceptor is shortened.

With respect to such a phenomenon, an action of avoiding direct influence of the gas onto the photoreceptor by efficiently discharging and replacing a gas around a corona charging unit, and an action of preventing deterioration by adding an antioxidant and a stabilizer to the photosensitive layer are proposed.

For example, Japanese Patent Application Laid-open Publication No. 62-105151 discloses adding an antioxidant having a triazine ring in its molecule and a hindered phenol backbone to a photosensitive layer, and Japanese Patent Application Laid-open Publication No. 63-18355 discloses adding a specific hindered amine to the photosensitive layer. Also, Japanese Patent Application Laid-open Publication No. 63-4238, Japanese Patent Application Laid-open Publication No. 63-216055, and Japanese Patent Application Laid-open Publication No. 3-172852 disclose adding trialkyl amine or aromatic amine to the photosensitive layer, and further Japanese Patent Application Laid-open Publication No. 5-158258 discloses adding an amine dimer to the photosensitive layer, however, all of these are not still sufficient.

That is, an effect of ozone resistance has not been sufficiently achieved by such as conventional arts, and it is a current state of art that there still remains an adverse effect in a practical use that the electrophotographic characteristic such as sensitivity and residual potential are impaired by addition of such antioxidant or the like. Therefore, a proposal of a novel material capable of improving an ozone resistance and having no adverse effect in the aspect of the electrophotographic characteristic is desired.

### SUMMARY OF THE INVENTION

Therefore, it is an object of the present invention to provide a novel triamine compound which can be used for providing the photoreceptor having an excellent effect of the ozone resistance and no adverse effect on other characteristic aspect and a method for producing the same, as well as the photoreceptor using the triamine compound and the image forming apparatus having the same.

Inventors of the present application made diligent efforts and unexpectedly found that a specific triamine compound has an excellent ozone resistance, and has no adverse effect in the aspect of the electrophotographic characteristic, and also is very useful for the photoreceptor and the image forming apparatus having the same, and finally accomplished the present invention.

According to the present invention, there is provided an electrophotographic photoreceptor, which is the electrophotographic photoreceptor including a monolayer type photosensitive layer containing the charge generating material and the charge transporting material, and an optional surface protective layer laminated on a conductive supporting member made of a conductive material, or which is the electrophotographic photoreceptor including a laminate type photosensitive layer having the charge generating layer containing the charge generating material and the charge transporting layer containing the charge transporting material laminated in this order, and the optional surface protective layer laminated on the conductive supporting member made of the conductive material, wherein

i) when the surface protective layer is not formed on a surface of the monolayer type photosensitive layer or the laminate type photosensitive layer, the charge transporting layer of the monolayer type photosensitive layer or the laminate type photosensitive layer contains a triamine compound represented by General formula (1) described below:

ii) when the surface protective layer is formed on respective surface of the monolayer type photosensitive layer or the laminate type photosensitive layer, both of the monolayer type photosensitive layer and the surface protective layer, or both of the charge transporting layer of the laminate type photosensitive layer and the surface protective layer contain the triamine compound represented by General formula (1) described below; or

the monolayer type photosensitive layer or the surface protective layer of the laminate type photosensitive layer contains the triamine compound represented by General formula (1) described below:

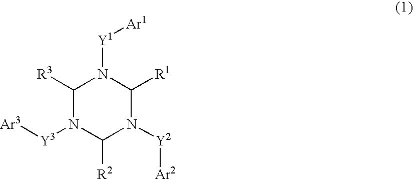

wherein, Ar¹, Ar² and Ar³, which are the same or different with each other, represent an optionally substituted aryl group, an optionally substituted cycloalkyl group, an optionally substituted hetero atom containing cycloalkyl group or an optionally substituted monovalent heterocyclic residue;

Y¹, Y² and Y³, which are the same or different with each other, represent an optionally substituted chained alkylene group; and

R¹, R² and R³, which are the same or different, represent an optionally substituted alkyl group, an optionally substituted aralkyl group, or a hydrogen atom.

In addition, according to the present invention, there is provided an image forming apparatus including the electrophotographic photoreceptor; a charging means that charges the electrophotographic photoreceptor; a light exposing means that exposes the charged electrophotographic photoreceptor to light; and a developing means that develops the electrostatic latent image formed by the light exposure.

Further, according to the present invention, there is provided an image forming apparatus, wherein the charging means is contact charging.

Further, according to the present invention, there is provided a triamine compound represented by Formula (4):

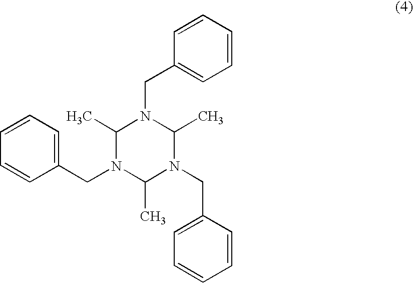

Further, according to the present invention, there is provided a triamine compound represented by Formula (5):

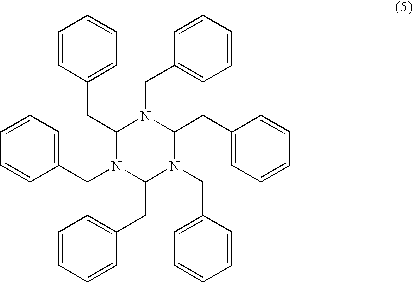

Further, according to the present invention, there is provided a method for producing a triamine compound, wherein compounds represented by the above Formulas (4) and (5) are obtained by causing an amine compound represented by Formula (6):

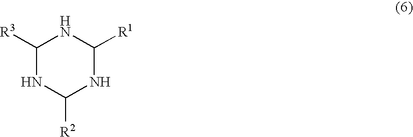

wherein R¹, R² and R³ represent a methyl group or a benzyl group, and a chloro compound represented by Formula (7):

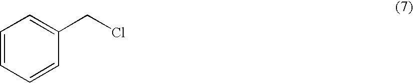

to react in the presence of an organic amine base.

The triamine compound of the present invention has the excellent effect of the ozone resistance and exerts no adverse effect in the aspect of the electrophotographic characteristic by being contained in each photosensitive layer containing the organic photoconductive material, so that it is preferable as a compound being used together with the organic photoconductive material.

Therefore, by causing the triamine compound according to the present invention to be contained, for example, in the photosensitive layer of the photoreceptor, it is possible to provide a photoreceptor having an improved effect of the ozone resistance, and at the same time an excellent durability and an environmental stability.

Further, the photoreceptor of the present invention is able to provide an image of high quality by its excellent effect of the ozone resistance even when it is used in a high-speed electrophotographic process.

Therefore, by using the photoreceptor according to the present invention, it is possible to form the image of high quality with excellent ozone resistance even after the repeated usage over a long term.

Further, since the photoreceptor according to the present invention contains the triamine compound according to the present invention in the photosensitive layer, it is excellent in effect of the ozone resistance, and is excellent in an aspect of a photoreceptor stop memory phenomenon in association with elongated lifetime of the photoreceptor.

Therefore, in the image forming apparatus according to the present invention, it is possible to stably form the image of high quality having no image defect for a long term in various environments.

Further, since the photoreceptor according to the present invention can provide the image of high quality even in the high-speed electrophotographic process, it is possible to increase an image forming speed in the image forming apparatus according to the present invention.

Further, according to the method for producing the triamine compound of the present invention, it is possible to conveniently obtain the triamine compound in a form of a crystal by causing a corresponding halogenated compound and an amine compound to react in a nonaqueous solvent under heating reaction, uniquely in the presence of the organic amine base.

Therefore, the compound obtained according to such production method is completely free from contamination of a metal compound which may cause adverse effects in the aspect of electrophotographic characteristic, and has no need of extracting operation such as liquid separation process, and hence the compound with high purity can be obtained by a simple operation.

### BRIEF DESCRIPTION OF THE DRAWINGS

FIG. 1 is a schematic view showing a configuration of an essential part of a monolayer type photoreceptor of the present invention;

FIG. 2 is a schematic view showing a configuration of essential part of a monolayer type photoreceptor of the present invention;

FIG. 3 is a schematic view showing a configuration of essential part of a monolayer type photoreceptor of the present invention;

FIG. 4 is a schematic view showing a configuration of essential part of a monolayer type photoreceptor of the present invention;

FIG. 5 is a schematic view showing a configuration of essential part of a laminate type photoreceptor of the present invention;

FIG. 6 is a schematic view showing a configuration of essential part of a laminate type photoreceptor of the present invention;

FIG. 7 is a schematic view showing a configuration of essential part of a laminate type photoreceptor of the present invention;

FIG. 8 is a schematic view showing a configuration of essential part of a laminate type photoreceptor of the present invention; and

FIG. 9 is a schematic side view showing a configuration of an image forming apparatus of the present invention.

### DETAILED DESCRIPTION OF THE INVENTION

The photoreceptor of the present invention is the electrophotographic photoreceptor including the monolayer type photosensitive layer containing the charge generating material and the charge transporting material, and the optional surface protective layer laminated on the conductive supporting member made of the conductive material, or the electrophotographic photoreceptor including the laminate type photosensitive layer having the charge generating layer containing the charge generating material and a charge transporting layer containing a charge transporting material laminated in this order, and the optional surface protective layer laminated on the conductive supporting member made of the conductive material, wherein

i) when the surface protective layer is not formed on the surface of the monolayer type photosensitive layer or the laminate type photosensitive layer, the charge transporting layer of the monolayer type photosensitive layer or the laminate type photosensitive layer contains the triamine compound represented by the General formula (1):

ii) when the surface protective layer is formed on respective surface of the monolayer type photosensitive layer or the laminate type photosensitive layer, both of the monolayer type photosensitive layer and the surface protective layer, or both of the charge transporting layer of the laminate type photosensitive layer and the surface protective layer contain the triamine compound represented by the General formula (1); or

the monolayer type photosensitive layer or the surface protective layer of the laminate type photosensitive layer contains the triamine compound represented by the General formula (1).

Among the triamine compounds represented by the General formula (1), from view points of a chemical stability such as degradation or deterioration as chemicals, an easiness of availability of a material, an easiness of production, a height of yield, a production cost, or the like, a triamine compound wherein Y¹, Y² and Y³ in the General formula (1) are chained alkylene groups, namely the triamine compound represented by General formula (2):

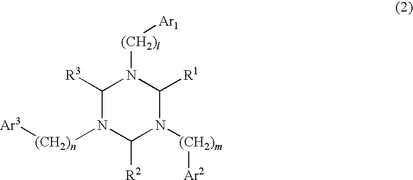

wherein Ar¹, Ar², Ar³, R¹, R² and R³ are synonymous as defined in the General formula (1); n, m and l, which are the same or different with each other, represent an integer of 1 to 3 is preferred, and a triamine compound wherein Y¹, Y² and Y³ in the General formula (1) are methylene groups, namely the triamine compound represented by General formula (3):

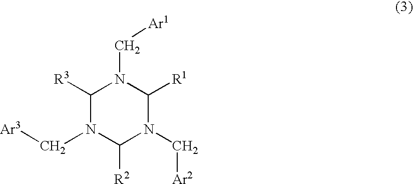

wherein Ar¹, Ar², Ar³, R¹, R² and R³ are synonymous as defined in the General formula (1) is particularly preferred.

Now, substituents in the General formulas (1), (2) and the Formula (3) will be described.

As the optionally substituted aryl group of Ar¹, Ar² and Ar³, for example, an alkyl group having 1 to 4 carbon atoms, an alkoxy group having 1 to 4 carbon atoms, a dialkyl amino group having 2 to 6 carbon atoms, and an aryl group which may be substituted with a halogen atom can be included.

Specific examples include such as a phenyl group, a tolyl group, a xylyl group, a methoxyphenyl group, a methyl methoxphenyl group, a t-butylphenyl group, a 4-diethylaminophenyl group, a 4-chlorophenyl group, a 4-fluorophenyl group, a naphthyl group and a methoxynaphthyl group, and among them, the phenyl group, the tolyl group, the methoxyphenyl group and the naphthyl group are particularly preferred.

As the optionally substituted cycloalkyl group of Ar¹, Ar² and Ar³, for example, a cycloalkyl group which may be substituted with an alkyl group having 1 to 4 carbon atoms can be included.

Specific examples include such as a cyclohexyl group, a cyclopentyl group and a 4,4-dimethylcyclohexyl group, and among them, the cyclohexyl group is particularly preferred.

As the optionally substituted hetero atom containing cycloalkyl group of Ar¹, Ar² and Ar³, for example, a tetrahydrofuryl group, a tetramethyltetrahydrofuryl group and the like can be included.

As the optionally substituted monovalent heterocyclic residue of Ar¹, Ar² and Ar³, for example, a monovalent heterocyclic residue which may be substituted with an alkyl group having 1 to 4 carbon atoms can be included.

Specific examples include such as a furyl group, a 4-methylfuryl group, a benzofuryl group, and a benzothiophenyl group, and among them, the furyl group and the benzofuryl group are particularly preferred.

As the optionally substituted chained alkylene group of Y¹, Y² and Y³, for example, an alkaline group which may be substituted with an alkyl group having 1 to 4 carbon atoms can be included.

Specific examples include such as a methylene group, an ethylene group, a propylene group, and a 2,2-dimethylpropylene group, and among them, the methylene group and the ethylene group are particularly preferred.

As the optionally substituted alkyl group of R¹, R² and R³, for example, an alkyl group having 1 to 4 carbon atoms, an alkoxy group having 1 to 4 carbon atoms, and an alkyl group which may be substituted with a halogen atom can be included.

Specifically, a methyl, an ethyl, an n-propyl, an isopropyl, a t-butyl, a trifluoromethyl, a 2-fluoroethyl, a 2,2,2-trifluoroethyl and a 1-methoxyethyl groups are particularly preferred.

As the optionally substituted aralkyl group of R¹, R² and R³, for example, an alkyl groups having 1 to 4 carbon atoms, an alkoxy group having 1 to 4 carbon atoms, and an aralkyl group which may be substituted with a halogen atom can be included.

Specifically, benzyl, 4-methylbenzyl, 4-methoxybenzyl, 3-ethylbenzyl and 1-naphthylmethyl are particularly preferred.

Specific examples of the triamine compound according to the present invention will be shown below.

TABLE 1

| | |
|---|---|
| | (1) |
| 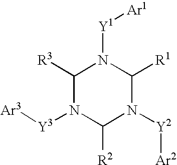 | |

| Ex- | | | | | | | | | |
|---|---|---|---|---|---|---|---|---|---|
| emplary | | | | | | | | | |
| com- | | | | | | | | | |
| pound | | | | | | | | | |
| No. | Ar¹ | Ar² | Ar³ | R¹ | R² | R³ | Y¹ | Y² | Y³ |
| 1 | 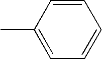 | 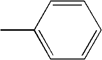 | 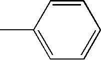 | -Me | -Me | -Me | -M- | -M- | -M- |
| 2 | 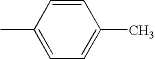 | 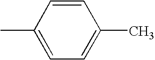 | 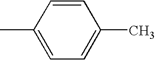 | -Me | -Me | -Me | -M- | -M- | -M- |
| 3 | 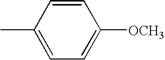 | 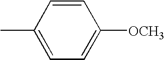 | 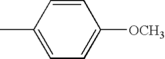 | -Me | -Me | -Me | -M- | -M- | -M- |
| 4 | 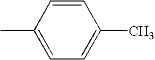 | 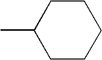 | 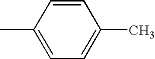 | -Me | -Me | -Me | -M- | -M- | -M- |
| 5 | 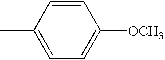 | 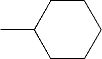 | 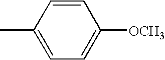 | -Me | -Me | -Me | -M- | -M- | -M- |
| 6 | 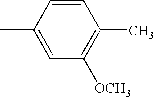 | 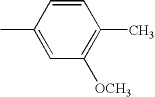 | 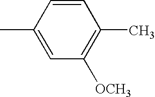 | -Me | -Me | -Me | -M- | -M- | -M- |
| 7 | 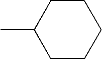 | 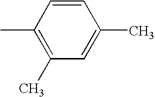 | 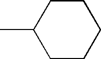 | -Me | -Me | -Me | -M- | -M- | -M- |
| 8 | 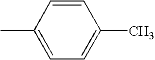 | 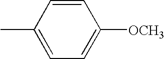 | 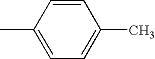 | -Me | -Me | -Me | -M- | -M- | -M- |
| 9 | 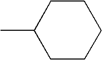 | 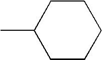 | 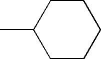 | -Me | -Me | -Me | -M- | -M- | -M- |
| 10 | 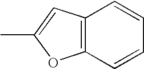 | 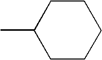 | 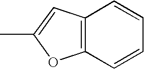 | -Me | -Me | -Me | -M- | -M- | -M- |
| 11 | 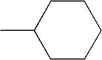 | 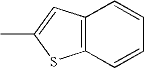 | 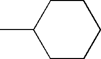 | -Me | -Me | -Me | -M- | -M- | -M- |
| 12 | 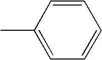 | 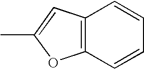 | 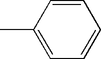 | -Me | -Me | -Me | -M- | -M- | -M- |
| 13 | 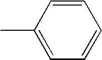 | 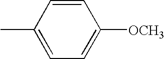 | 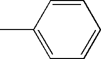 | -Me | -Me | -Me | -M- | -M- | -M- |
| 14 | 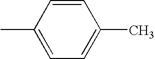 | 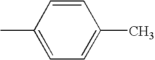 | 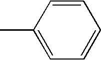 | -Me | -Me | -Me | -M- | -M- | -M- |
| 15 | 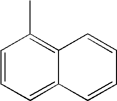 | 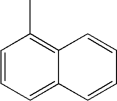 | 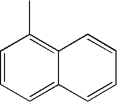 | -Me | -Me | -Me | -M- | -M- | -M- |
| 16 | 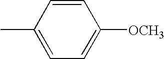 | 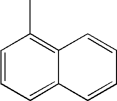 | 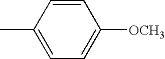 | -Me | -Me | -Me | -M- | -M- | -M- |
| 17 | 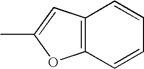 | 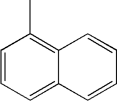 | 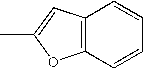 | -Me | -Me | -Me | -M- | -M- | -M- |
| 18 | 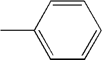 | 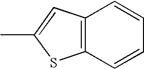 | 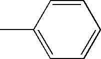 | -Me | -Me | -Me | -M- | -M- | -M- |
| 19 | 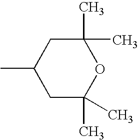 | 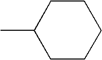 | 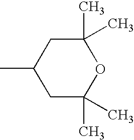 | -Me | -Me | -Me | -M- | -M- | -M- |
| 20 | 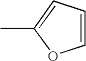 | 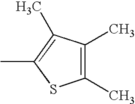 | 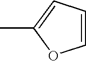 | -Me | -Me | -Me | -M- | -M- | -M- |
| 21 | 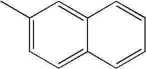 | 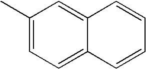 | 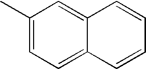 | -Me | -Me | -Me | -M- | -M- | -M- |
| 22 | 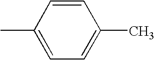 | 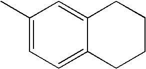 | 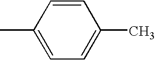 | -Me | -Me | -Me | -M- | -M- | -M- |
| 23 | 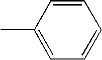 | 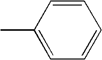 | 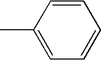 | -Me | -Me | -Me | -E- | -E- | -E- |
| 24 | 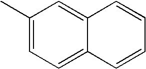 | 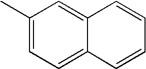 | 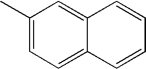 | -Me | -Me | -Me | -E- | -E- | -E- |
| 25 | 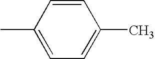 | 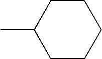 | 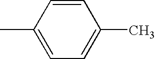 | -Me | -Me | -Me | -E- | -E- | -E- |
| 26 | 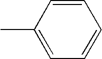 | 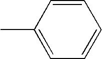 | 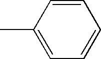 | -Me | -Me | -Me | -P- | -P- | -P- |
| 27 | 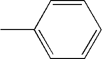 | 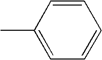 | 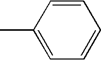 | -Me | -Me | -Me | -DMP- | -DMP- | -DMP- |
| 28 | 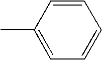 | 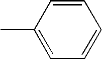 | 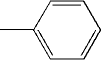 | -Me | -Me | -Me | -M- | -E- | -E- |
| 29 | 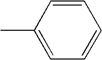 | 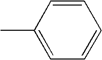 | 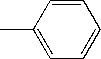 | -Me | -Me | -Me | -M- | -M- | -DMP- |
| 30 | 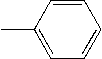 | 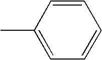 | 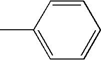 | -Me | -Me | -Me | -M- | -E- | -P- |
| 31 | 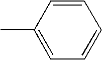 | 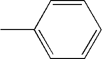 | 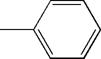 | H | H | H | -M- | -M- | -M- |
| 32 | 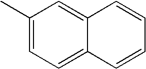 | 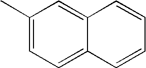 | 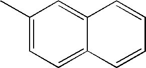 | H | H | H | -M- | -M- | -M- |
| 33 | 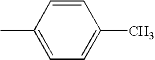 | 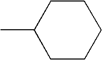 | 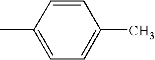 | H | H | H | -M- | -M- | -M- |
| 34 | 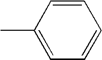 | 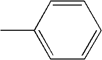 | 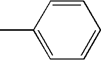 | -Et | -Et | -Et | -M- | -M- | -M- |
| 35 | 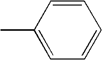 | 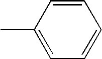 | 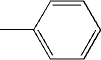 | -TFM | -TFM | -TFM | -M- | -M- | -M- |
| 36 | 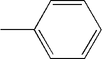 | 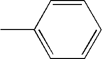 | 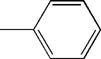 | -iBu | -iBu | -iBu | -M- | -M- | -M- |
| 37 | 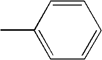 | 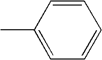 | 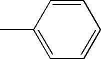 | 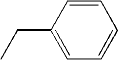 | 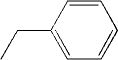 | 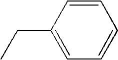 | -M- | -M- | -M- |
| 38 | 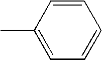 | 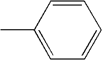 | 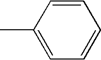 | 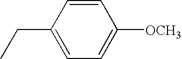 | 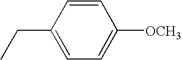 | 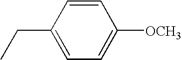 | -M- | -M- | -M- |
| 39 | 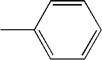 | 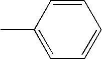 | 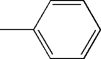 | -Me | -Me | -Et | -M- | -M- | -M- |
| 40 | 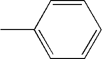 | 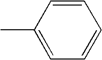 | 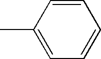 | H | H | 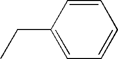 | -M- | -M- | -M- |
| 41 | 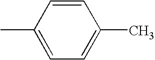 | 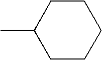 | 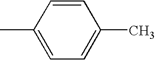 | -Me | -Me | -Et | -M- | -E- | -E- |

In the Table, -Me means the methyl group, -Et means the ethyl group, iBu means the isobutyl group and -TFM means the trifluoromethyl group, respectively.
In addition, -M- means the methylene group, -E- means the ethylene group, -P- means the propylene group, and -DMP- means a 2,2-dimethyltrimethylene group.

Triamine compound (I) of the present invention may be produced by a method shown in a reaction scheme described below.

That is, an objective product may be produced by heating the amine compound represented by the General formula (6) and a halogen compound shown by General formula (8) in the presence of an organic amine base.

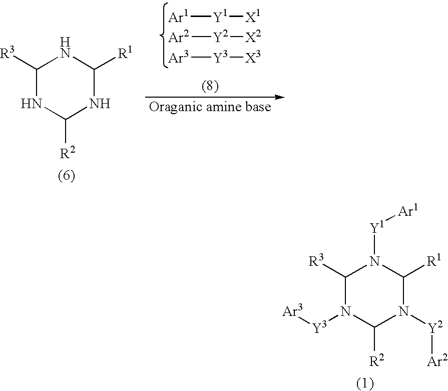

wherein, Ar¹, Ar², Ar³, Y¹, Y², Y³, R¹, R² and R³ are synonymous as defined in the General formula (1), and X¹, X² and X³ represent a halogen atom.

As the halogen atom of X¹, X² and X³ in the above reaction formula, a chlorine atom, a bromine atom and an iodine atom can be included, and among them, the chlorine atom and the bromine atom are particularly preferred.

The above reaction may be carried out, for example, in a following manner.

The amine compound (6) and the halogen compound (8) are dissolved or dispersed in a solvent, and added with the organic amine base, and then stirred under heating. After completion of reaction, a precipitate is separated by filtering, and allowed to recrystallize in a single or a mixed solvent system such as ethanol, methanol and ethyl acetate. In this manner, the objective product can be obtained conveniently with high yield and high purity.

As the solvent used in the above reaction, any solvents that are inert in the above reaction and are capable of dissolving or dispersing a reaction substrate and the organic amine base may be used without any particular limitation. Specific examples include aromatic hydrocarbons such as toluene and xylene; ethers such as diethyl ether, tetrahydrofuran, ethyleneglycol dimethylether and 1,4-dioxane; amides such as N,N-dimethyl formamide; and sulfoxides such as dimethyl sulfoxide, and these may be used alone or as a mixed solvent.

Here, an amount of the solvent used is not particularly limited, and the amount that allows smooth proceeding of reaction may be appropriately set depending on reaction conditions such as an amount of the reaction substrate used, a reaction temperature and a reaction time.

As the organic amine base, for example, N,N-diisopropyl ethylamine, N,N-dimethylaminopyridine, and 1,4-diazabicycloundecene can be included.

A ratio of the amine compound (6) and the halogen compound (8) used is not particularly limited, however, in consideration of efficiency of reaction, it is preferable that about 3.0 to 3.6 equivalents of the total halogen compound (8) is used, relative to 1.0 equivalent of secondary amine compound (6).

In addition, a ratio of the halogen compound (8) and the organic amine base is not particularly limited, however, in consideration of efficiency of the reaction, it is preferable that about 1.05 to 2.0 equivalents of the organic amine base is used, relative to 1.0 equivalent of the halogen compound (8).

Further, the heating temperature and the reaction time are not particularly limited, however, in consideration of efficiency of the reaction, it is preferred that the reaction is allowed to last at 60 to 120° C. for 2 to 8 hours depending on the solvent being used.

The triamine compound according to the present invention has a characteristic of the excellent ozone resistance.

Therefore, the photoreceptor containing the triamine compound of the present invention in the photosensitive layer (in particular, charge transporting layer) has the excellent electrophotographic characteristic, and is difficult to be influenced by ozone generating in the system, and nitrogen oxides, so that the stable characteristic and the image quality are kept even after the repeated usage, and very high durability can be achieved.

In particular, the triamine compound (4) represented by Formula (4);

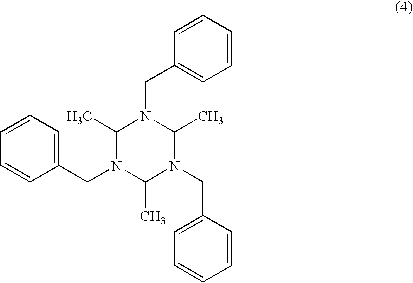

and the triamine compound (5) represented by Formula (5)

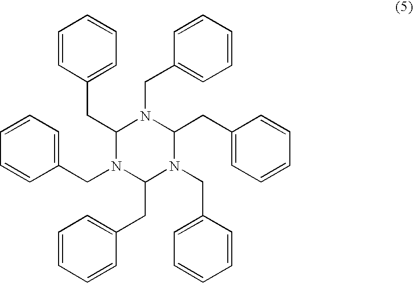

are novel compounds.

The above triamine compounds (4) and (5) may be produced by causing the amine compound represented by the General formula (6):

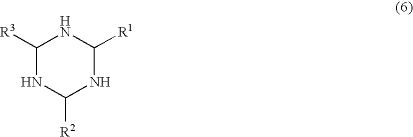

wherein, R¹, R² and R³ represent the methyl group or the benzyl group and the benzyl chloride (chloro compound) represented by the Formula (7):

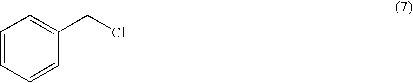

to react in the presence of the organic amine base.

Next, a configuration of the photoreceptor according to the present invention will be specifically described.

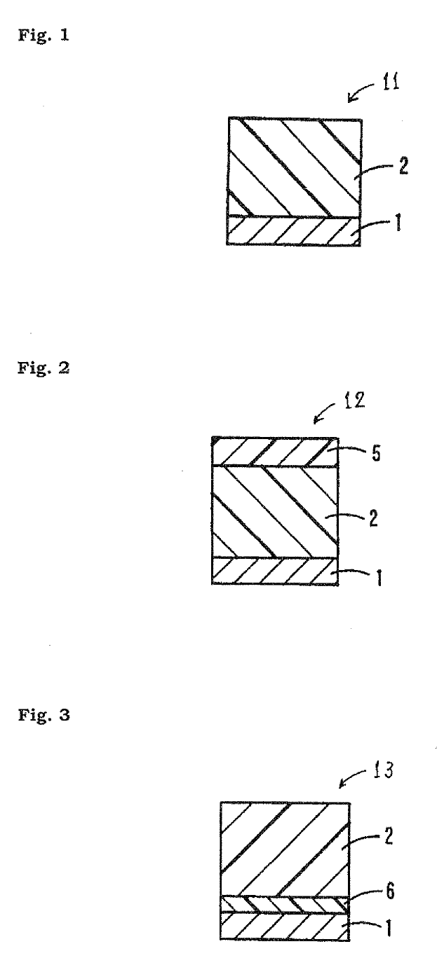

FIGS. 1 to 8 are schematic section views showing configurations of essential parts in the photoreceptors of the present invention.

FIGS. 1 to 4 are schematic section views showing configurations of essential parts of the monolayer type photoreceptors in which the photosensitive layer is the monolayer type photosensitive layer made up of a single layer.

In addition, FIGS. 5 to 8 are schematic section views showing configurations of essential parts of the laminate type photoreceptors (hereinafter, also referred to as a “function separated type photoreceptor”) in which the photosensitive layer is a laminate type photosensitive layer made up of the charge generating layer and the charge transporting layer. The photoreceptor of the present invention may be a reverse double layer type laminate structure in which the charge generating layer and the charge transporting layer are formed in reverse order, however, the aforementioned laminate type is preferred.

In a photoreceptor 11 of FIG. 1, a monolayer type photosensitive layer 2 is formed on a surface of a conductive supporting member 1.

In a photoreceptor 12 of FIG. 2, the monolayer type photosensitive layer 2 and a surface protective layer 5 are formed in this order on the surface of the conductive supporting member 1.

In a photoreceptor 13 of FIG. 3, an intermediate layer 6 and the monolayer type photosensitive layer 2 are formed in this order on the surface of the conductive supporting member 1.

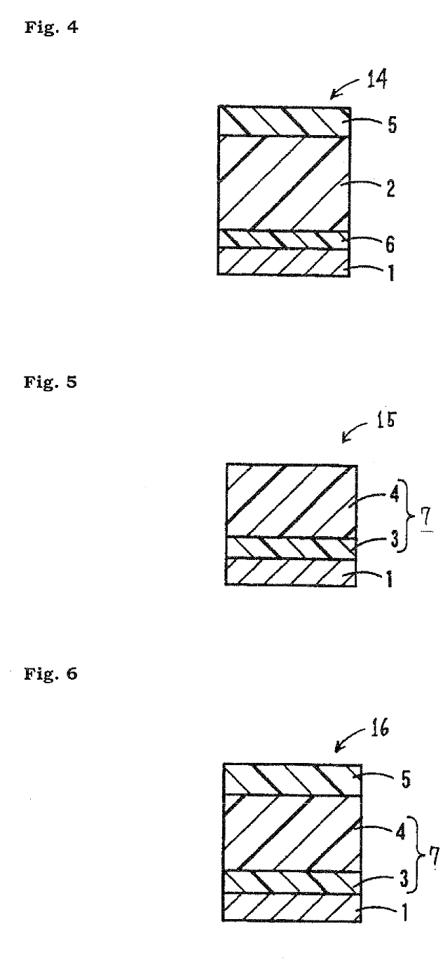

In a photoreceptor 14 of FIG. 4, the intermediate layer 6, the monolayer type photosensitive layer 2 and the surface protective layer 5 are formed in this order on the surface of the conductive supporting member 1.

In a photoreceptor 15 of FIG. 5, a laminate type photosensitive layer 7 in which a charge generating layer 3 and a charge transporting layer 4 are laminated in this order is formed on the surface of the conductive supporting member 1.

In a photoreceptor 16 of FIG. 6, the laminate type photosensitive layer 7 in which the charge generating layer 3 and the charge transporting layer 4 are laminated in this order, and the surface protective layer 5 are formed in this order on the surface of the conductive supporting member 1.

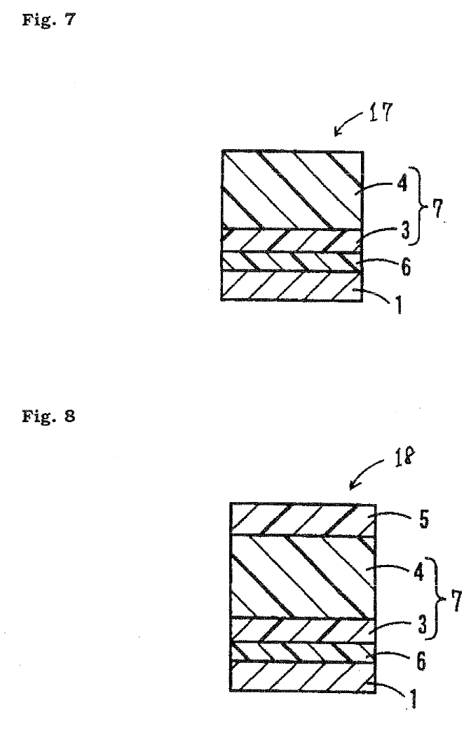

In a photoreceptor 17 of FIG. 7, the intermediate layer 6, and the laminate type photosensitive layer 7 in which the charge generating layer 3 and the charge transporting layer 4 are laminated in this order are formed in this order on the surface of the conductive supporting member 1.

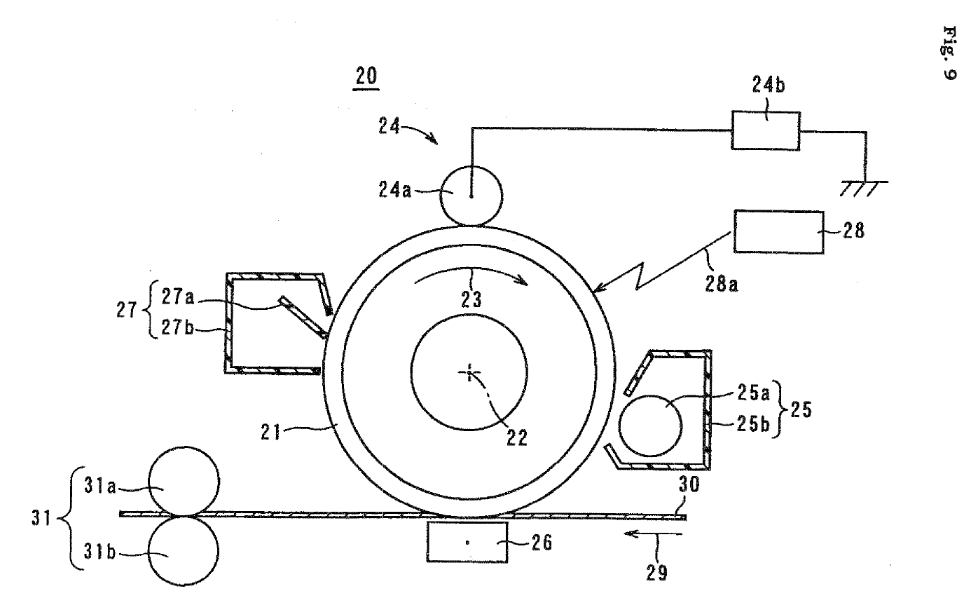

In a photoreceptor 18 of FIG. 8, the intermediate layer 6, the laminate type photosensitive layer 7 in which the charge generating layer 3 and the charge transporting layer 4 are laminated in this order, and the surface protective layer 5 are formed in this order on the surface of the conductive supporting member 1.

### [Conductive Supporting Member 1 (Base Pipe for Photoreceptor)]

The material forming the conductive supporting member is not particularly limited insofar as used in the art.

Specific examples include a metal material such as aluminum, an aluminum alloy, copper, zinc, stainless steel and titanium; a polymer material such as polyethylene terephthalate, polyamide, polyester, polyoxymethylene and polystyrene; those obtained by laminating metal foil, or by vapor depositing the metal material, or by vapor-depositing or applying a layer of conductive compound such as conductive polymer, tin oxide, or indium oxide, on a surface of a base material of a hard paper, a glass and the like.

A form of the conductive supporting member is not limited to a sheet form as shown in FIGS. 1 to 8, and may be such as a columnar, a cylindrical or a seamless belt form.

The surface of the conductive supporting member 1 may be subjected to anode oxidation film treatment, surface treatment by chemicals, hot water and the like, coloring treatment, and irregular reflection treatment surface roughening treatment, as necessary as far as such treatment will not influence on the image quality.

The irregular reflection treatment is particularly effective when the photoreceptor according to the present invention is used in the electrophotographic process where the laser is used as the exposure light source. That is, in the electrophotographic process where the laser is used as the exposure light source, since wavelength of laser beam is uniform, the laser beam reflected on the surface of the photoreceptor and the laser beam reflected inside the photoreceptor interfere with each other, and a fringe resulting from the interference may appear in image to lead an occurrence of an image defect. Consequently, by subjecting the surface of the conductive supporting member to the irregular reflection treatment, the image defect by the interference between the laser beams having uniform wavelength can be prevented.

### [Monolayer Type Photosensitive Layer 2]

The monolayer type photosensitive layer 2 contains the charge generating material, the charge transporting material, the triamine compound of the present invention, and a binder resin.

The charge generating material is able to generate an electric charge by absorbing light.

As the charge generating material, any compounds used in the art may be used.

Specific examples include an organic pigment or dye such as an azoic pigment (a monoazoic pigment, a bisazoic pigment, a triazoic pigment and the like), an indigoic pigment (indigo, thioindigo and the like), a perylenic pigment (perylene imide, perylenic acid anhydride and the like), a polycyclic quinonic pigment (anthraquinone, pyrenequinone and the like), a phthalocyaninic pigment (metal phthalocyanine, X-type nonmetallic phthalocyanine and the like), a squarylium dye, pyrylium salts, thiopyrylium salts, and a triphenylmethanic dye, and an inorganic material such as selenium and amorphous silicon. These charge generating materials may be used alone or in combination of two or more kinds.

Among these charge generating materials, the phthalocyaninic pigment such as the metallic phthalocyanine and the X-type nonmetallic phthalocyanine are preferred, and oxotitanium phthalocyanine is particularly preferred.

Since the phthalocyaninic pigment has a high charge generating efficiency and a high charge injecting efficiency, they generate a large amount of charges by absorbing light, and the generated charges will be efficiently injected into the charge transporting material contained in the monolayer type photosensitive layer and smoothly transported, without being accumulated in its molecule. Therefore, the photoreceptor having a high sensitivity and a high resolution can be obtained. This effect also applies to the laminate type photoreceptor as will be described later.

The charge generating material may be used in combination with a sensitizing dye.

As such sensitizing dye, for example, a triphenylmethanic dye represented by such as methyl violet, crystal violet, night blue and victoria blue; an acridine dye represented by such as erythrosine, rhodamine B, rhodamine 3R, acridine orange and flapeosine; a thiadine dye represented by such as methylene blue and methylene green; an oxazine dye represented by such as capri blue and meldola blue; a cyanine dye; a styryl dye, a pyrylium salt dye and a thiopyrylium salt dye can be exemplified.

The charge transporting material is able to receive and transport an electric charge generated in the charge generating material, and includes a hole transporting substance and an electron transporting substance.

As the hole transporting substance, any compounds used in the art may be used.

Specific examples include a carbazole derivative, a pyrene derivative, an oxazole derivative, an oxathiazole derivative, a thiazole derivative, a thiadiazole derivative, a triazole derivative, an imidazole derivative, an imidazolone derivative, an imidazolidine derivative, a bisimidazolidine derivative, a styryl compound, a hidrazone compound, a polycyclic aromatic compound, an indole derivative, a pyrazoline derivative, an oxazolone derivative, a benzimidazole derivative, a quinazoline derivative, a benzofuran derivative, an acridine derivative, a phenadine derivative, an aminostilbene derivative, a triarylamine derivative, a triarylmethane derivative, a phenylene diamine derivative, a stilbene derivative, an enamine derivative, a benzidine derivative, a polymer having in its main chain or side chain, a group derived from these compounds (poly-N-vinylcarbazole, poly-1-vinylpyrene, an ethylcarbazole-formaldehyde resin, a triphenylmethane polymer, poly-9-vinylanthracene and the like), and polysilane.

As the electron transporting substance, any compounds used in the art may be used.

Specific examples include an organic compound such as a benzoquinone derivative, a tetracyano ethylene derivative, a tetracyano quinomethane derivative, a fluorenone derivative, a xanthone derivative, a phenanthraquinone derivative, a phthalic anhydride derivative, and a diphenoquinone derivative, and an inorganic material such as amorphous silicon, amorphous selenium, tellurium, a selenium-tellurium alloy, cadmium sulfide, antimony sulfide, zinc oxide and zinc sulfide. These charge transporting materials may be used alone or in combination of two or more kinds.

As the binder resin, for example, the resin that is used for the purpose of improving the mechanical strength, the durability and the like of the monolayer type photosensitive layer and have a binding property used in the art may be used, and one having an excellent compatibility with the triamine compound of the present invention is preferred.

Specific examples include a thermoplastic resin such as polymethyl methacrylate, polystyrene, a vinylic resin of polyvinyl chloride and the like, polycarbonate, polyester, polyester carbonate, polysulfone, polyarylate, polyamide, a methacryl resin, an acryl resin, polyether, polyacrylamide, and polyphenylene oxide; a thermosetting resin such as a phenoxy resin, an epoxy resin, a silicone resin, polyurethane, a phenol resin, an alkyd resin, a melamine resin, a phenoxy resin, polyvinyl butyral, and polyvinylformal, a partially cross-linked product of these resins, and a copolymer resin containing two or more of constituent units contained in these resins (an insulating resin of a vinyl chloride-vinyl acetate copolymer resin, a vinyl chloride-vinyl acetate-maleic anhydride copolymer resin, an acrylonitrile-styrene copolymer resin and the like). These binder resins may be used alone or in combination of two or more kinds.

Among these resins, the polystyrene, the polycarbonate, the polyarylate and the polyphenylene oxide are preferable because they have particularly excellent compatibility with the triamine compound of the present invention, an excellent electric insulating property as having a volume resistance of 10¹³Ω or higher, and have an excellent film formability, a potential characteristic or the like, and the polycarbonate can be particularly preferably used.

A ratio of the charge transporting material and the triamine compound of the present invention used is not particularly limited, however, it is preferred that when a weight of the charge transporting material is referred to as “A”, and a weight of the triamine compound is referred to as “B”, the ratio A/B is 100/0.1 or more and 100/20 or less.

When an amount of the triamine compound of the present invention used is less than 0.1 parts by weight relative to 100 parts by weight of the charge transporting material, the effect may be very small.

On the other hand, when an amount of the triamine compound of the present invention used exceeds 20 parts by weight relative to 100 parts by weight of the charge transporting material, a relative quantitative ratio with respect to the charge transporting material becomes high, so that a phenomenon of decrease in sensitivity and the like may occur.

Further, the monolayer type photosensitive layer may contain an additive such as antioxidant used in the art. Such additive is desirable because it improves stability as an application liquid for forming the photosensitive layer, elongates a liquid life time, and at the same time, also reduces oxidized impurities in the photoreceptor produced by the application liquid to improve the durability.

As the antioxidant, for example, a hindered phenol derivative, a hindered amine derivative and the like may be included.

A ratio of the charge transporting material and the antioxidant used is not particularly limited, however, 0.1 to 10 parts by weight, relative to 100 parts by weight of the charge transporting material is preferred. When the use amount of the antioxidant is less than 0.1 part by weight, the stability of the application liquid for forming the photosensitive layer that will be described later, and the effect of improving the durability of the photoreceptor may be insufficient, while when it exceeds 10 parts by weight, the adverse effect may be exerted on the electric characteristic of the photoreceptor.

A ratio of the triamine compound of the present invention, the charge generating material, the charge transporting material, and the additive and the binder added as necessary is not particularly limited, however, it is preferred that the binder resin occupies about 40 to 60% by weight in a total amount.

When a proportion of the binder resin is less than 40% by weight, the film strength of the monolayer type photosensitive layer may be deteriorated, whereas when the proportion of the binder resin exceeds 60% by weight, the function of the monolayer type photosensitive layer may be deteriorated.

The monolayer type photosensitive layer 2 may be formed by preparing the application liquid for forming the photosensitive layer by dissolving or dispersing the triamine compound of the present invention, the charge generating material, the charge transporting material and the binder resin, as well as the additive such as antioxidant as necessary in an appropriate organic solvent, and applying this application liquid on the surface of the conductive supporting member 1, or on the surface of the intermediate layer 6 formed on the conductive supporting member 1, followed by drying to remove the organic solvent.

More specifically, for example, by dissolving or dispersing constituting substance in a resin solution prepared by dissolving the binder resin in the organic solvent, the application liquid for forming the monolayer type photosensitive layer is prepared.

As the organic solvent, for example, aromatic hydrocarbons such as benzene, toluene, xylene, mesitylene, tetralin, diphenylmethane, dimethoxybenzene and dichlorobenzene; halogenated hydrocarbons such as dichloromethane, dichloroethane and tetrachloropropane; ethers such as tetrahydrofuran (THF), dioxane, dibenzylether, dimethoxymethyl ether and 1,2-dimethoxy ethane; ketones such as methylethyl ketone, cyclohexanone, acetophenone and isophorone; esters such as methyl benzoate, ethyl acetate, and butyl acetate; a sulfur containing solvent such as diphenylsulfide; a fluorine-based solvent such as hexafluoroisopropanol; and an aprotic polar solvent such as N,N-dimethylformamide and N,N-dimethylacetamide, and the like can be included. These may be used alone or as a mixed solvent. A mixed solvent prepared by adding alcohols, acetonitrile or methylethyl ketone to the solvent as described above may be used.

Before dissolving or dispersing the constituting substances in the resin solution, the charge generating material and other additive may be pre-ground.

A pre-grinding may be conducted by using a commonly used grinder such as a ball mill, a sand mill, an attritor, a vibration mill or an ultrasonic disperser.

The constituting substances may be dissolved or dispersed in the resin solution, for example, by a commonly used disperser such as a paint shaker, the ball mill, or the sand mill. At this time, it is preferred to appropriately set a dispersing condition to prevent the impurities from generating from the members constituting a container and a disperser due to abrasion, to be mixed with the application liquid.

As an application method of the application liquid for forming the monolayer type photosensitive layer, a roll coating, a spray coating, a blade coating, a ring coating, a dip coating or the like can be included.

A film thickness of the monolayer type photosensitive layer is not particularly limited, however, 5 to 100 μm is preferred, and 10 to 50 μm is particularly preferred. When the film thickness of the monolayer type photosensitive layer is less than 5 μm, a charge retaining ability of the surface of the photoreceptor may be decreased, whereas when the film thickness of the monolayer type photosensitive layer exceeds 100 μm, a productivity of the photoreceptor may be deteriorated.

### [Laminate Type Photosensitive Layer 7]

The laminate type photosensitive layer 7 is made up of the charge generating layer 3 and the charge transporting layer 4.

### [Charge Generating Layer 3]

The charge generating layer 3 contains the charge generating material and a binder resin, and may optionally contain the triamine compound of the present invention.

As the charge generating material, one kind or two or more kinds of the charge generating material similar to those contained in the monolayer type photosensitive layer may be used.

As the binder resin, one kind or two or more kinds of the binder resin similar to those contained in the monolayer type photosensitive layer may be used.

A ratio of the charge generating material and the binder resin used is not particularly limited, however, it is preferred that a the total amount which is a sum amount of the charge generating material and the binder resin, the charge generating material occupies 10 to 99% by weight, and a remnant is the binder resin.

When a proportion of the charge generating material is less than 10% by weight, the sensitivity may be deteriorated, whereas when the proportion of the charge generating material exceeds 99% by weight, not only the film strength of the charge generating layer is impaired, but also the dispersivity of the charge generating material is deteriorated and bulky particles increase, and the surface charge in an area other than the part that is to be deleted by the light exposure decreases, so that the image defect, in particular, fogging in image, called a black spot where a fine black dot is formed by adhesion of toner on a white base, may often arise.

The charge generating layer may contain an appropriate amount of each of one kind or two or more kinds selected from a hole transporting material, an electron transporting material, the antioxidant, an dispersion stabilizer, a sensitizer and the like as necessary, in addition to the two essential components as described above. This improves a potential characteristic, and at the same time increases stability of an application liquid for forming the charge generating layer that will described later, so that it is possible to mitigate a fatigue and deterioration after the repeated usage of the photoreceptor and to improve the durability.

The charge generating layer 3 may be formed by preparing the application liquid for forming the charge generating layer by dissolving or dispersing the charge generating material, the binder resin and the other additive as necessary in the appropriate organic solvent, and applying the application liquid on the surface of the conductive supporting member 1, or on the surface of the intermediate layer 6 formed on the conductive supporting member 1, followed by drying to remove the organic solvent. More specifically, for example, by dissolving or dispersing the charge generating material and the other additive as necessary in the resin solution prepared by dissolving the binder resin in the organic solvent, the application liquid for forming the charge generating layer is prepared.

Other steps and other conditions are in accordance with the formation of the monolayer type photosensitive layer.

As the organic solvent, one kind or two or more kinds of the solvents similar to those used in the application liquid for forming the monolayer type photosensitive layer may be used.

A film thickness of the charge generating layer 3 is not particularly limited, however, 0.05 to 5 μm is preferred, and 0.1 to 1 μm is particularly preferred. When the film thickness of the charge generating layer is less than 0.05 μm, an efficiency of a light absorption is reduced and the sensitivity may be decreased, whereas when the film thickness of the charge generating layer exceeds 5 μm, a charge transportation inside the charge generating layer is a rate-determining stage in a course of deleting charges on the surface of the photoreceptor, and the sensitivity may be decreased.

### [Charge Transporting Layer 4]

The charge transporting layer 4 contains the charge transporting material, the triamine compound according to the present invention, and the binder resin.

As the triamine compound of the present invention, one kind or two or more kinds of the triamine compounds similar to those contained in the monolayer type photosensitive layer may be used.

As the charge transporting material, one kind or two or more kinds of the charge transporting materials similar to those contained in the monolayer type photosensitive layer may be used.

As the binder resin, one kind or two or more kinds of the binder resins similar to those contained in the monolayer type photosensitive layer may be used.

The ratio of the charge transporting material and the triamine compound of the present invention used is similar to that in the monolayer type photosensitive layer.

The ratio of the charge generating material and the binder resin used is similar to that in the monolayer type photosensitive layer.

The charge transporting layer may contain the additive such as antioxidant similar to that contained in the monolayer type photosensitive layer, as necessary, besides the above three essential components.

The charge transporting layer 4 may be formed by preparing an application liquid for forming the charge transporting layer by dissolving or dispersing the charge transporting material, the triamine compound of the present invention, the binder resin and the other additive as necessary in the appropriate organic solvent, and applying this application liquid on the surface of the charge generating layer 3, followed by drying to remove the organic solvent. More specifically, for example, the application liquid for forming the charge transporting layer is prepared by dissolving or dispersing the charge transporting material, the triamine compound of the present invention and the other additive as necessary in the resin solution prepared by dissolving the binder resin in the organic solvent.

Other steps and other conditions are in accordance with the formation of the monolayer type photosensitive layer.

A film thickness of the charge transporting layer 4 is not particularly limited, however, 5 to 50 μm is preferred, and 10 to 40 μm is particularly preferred. When the film thickness of the charge transporting layer is less than 5 μm, the charge retaining ability of the surface of the photoreceptor may be decreased, whereas when the film thickness of the charge transporting layer exceeds 50 μm, the resolution of the photoreceptor may be deteriorated.

### [Surface Protective Layer 5]

The surface protective layer 5 has a function of improving the durability of the photoreceptor, and contains the charge transporting material and the binder resin, and may optionally contain the triamine compound according to the present invention, and may optionally be provided on the surface of the monolayer type photosensitive layer or the laminate type photosensitive layer.

As the charge transporting material, one kind or two or more kinds of the charge transporting materials similar to those contained in the monolayer type photosensitive layer may be used.

As the binder resin, one kind or two or more kinds of the binder resins similar to those contained in the monolayer type photosensitive layer may be used.

The surface protective layer 5 may be prepared, for example, by preparing an application solution for forming the surface protective layer by dissolving or dispersing the charge transporting material, the binder resin and the like in the appropriate organic solvent, and applying the application liquid for forming the surface protective layer on the surface of the monolayer type photosensitive layer 2 or the laminate type photosensitive layer 7, followed by drying to remove the organic solvent.

As the organic solvent used herein, one kind or two or more kinds of the organic solvents similar to those used in formation of the photosensitive layer 2 may be used.

Other steps and other conditions are in accordance with the formation of the monolayer type photosensitive layer.

A film thickness of the surface protective layer 5 is not particularly limited, however, 0.5 to 10 μm is preferred, and 1 to 5 μm is particularly preferred. When the film thickness of surface protective layer 5 is less than 0.5 μm, a rubfastness of the surface of the photoreceptor becomes poor and the durability may be insufficient, whereas when it exceeds 10 μm, the resolution of the photoreceptor may be deteriorated.

### [Intermediate Layer 6]

Preferably, the photoreceptor of the present invention has an intermediate layer between the conductive supporting member and the monolayer type photosensitive layer or the laminate type photosensitive layer.

The intermediate layer has a function of preventing injection of the charge from the conductive supporting member into the monolayer type photosensitive layer or the laminate type photosensitive layer. That is, a reduction in chargeability of the monolayer type photosensitive layer or the laminate type photosensitive Layer is suppressed, and a reduction in the surface charge in the area other than the part to be deleted by light exposure is suppressed, and an occurrence of the image defect such as fogging is prevented. In particular, in the case of the image formation by a reversal development process, the occurrence of image fogginess called the black spot wherein the fine black dot of toner is formed on the white base part is prevented.

Further, the intermediate layer that covers the surface of the conductive supporting member is able to reduce a degree of roughness which is a defect of a surface of the conductive supporting member to be homogenization, thereby improving the film formability of the monolayer type photosensitive layer or the laminate type photosensitive layer, and improving an adhesion between the conductive supporting member, and the monolayer type photosensitive layer or the laminate type photosensitive layer.

The intermediate layer may be formed, for example, by preparing an application liquid for forming the intermediate layer by dissolving a resin material in the appropriate solvent, and applying this application liquid on the surface of the conductive supporting member, followed by drying to remove the organic solvent.

As the resin material, in addition to the binder resin similar to that contained in the monolayer type photosensitive layer, a natural polymer material such as casein, gelatin, polyvinyl alcohol and ethyl cellulose can be included, and one kind or two or more kind of them may be used.

As the solvent for dissolving or dispersing the resin material, for example, water, alcohols such as methanol, ethanol and butanol, grimes such as methyl carbitol and butyl carbitol, and a mixed solvent in which two or more kinds of these solvents are mixed can be included.

Other steps and other conditions are in accordance with the formation of the monolayer type photosensitive layer.

The application liquid for forming a intermediate layer may also contain metal oxide particles.

The metal oxide particles are able to easily regulate a volume resistance of the intermediate layer and further prevent injection of the charge into the monolayer type photosensitive layer or the laminate type photosensitive layer, and at the same time to keep the electric characteristic of the photoreceptor under various environments.

As the metal oxide particles, for example, titanium oxide, aluminum oxide, aluminum hydroxide, zinc oxide and the like can be included.

When taking a total content of the resin material and the metal oxide particles in the application liquid for forming the a intermediate layer as “C”, and a content of the solvent as “D”, a capacity ratio of both (C/D) is preferably 1/99 to 40/60 (weight ratio-0.01 to 0.67), and is particularly preferably 2/98 to 30/70 (weight ratio=0.02 to 0.43).

Further, a capacity ratio (E/F) between the content of resin material (E) and the content of metal oxide particles (F) is preferably 1/99 to 90/10 (weight ratio=0.01 to 9.0), and particularly preferably 5/95 to 70/30 (weight ratio=0.05 to 2.33).

A film thickness of the intermediate layer is not particularly limited, however, it is preferably 0.01 to 20 μm, and more preferably 0.1 to 10 μm. When the film thickness of the intermediate layer is less than 0.01 μm, a functionality as the intermediate layer is substantially lost, and a uniform surface of the conductive supporting member by covering the defect may not be obtained, whereas when the film thickness of the intermediate layer exceeds 20 μm, a uniform intermediate layer is difficult to be formed, and the sensitivity of the photoreceptor may be deteriorated.

When the constituting material of the conductive supporting member is aluminum, a layer containing alumite (alumite layer) may be formed as the intermediate layer.

The image forming apparatus of the present invention includes the photoreceptor of the present invention, the charging means that charges the photoreceptor, the light exposing means that exposes the photoreceptor to light, and the developing means that develops an electrostatic latent image formed by the light exposure.

Referring to drawings, the image forming apparatus of the present invention will be described, however, it will not be limited to a description described below.

FIG. 9 is a schematic side view showing a configuration of the image forming apparatus of the present invention.

An image forming apparatus 20 in FIG. 9 is composed of a photoreceptor of the present invention 21 (for example, either one of the photoreceptors 11 to 18 in FIG. 1 to 8), a charging means (charging unit) 24, a light exposing means 28, a developing means (developing unit) 25, a transferring unit 26, a cleaner 27, and a fixing unit 31. A reference numeral 30 denotes a transfer paper.

The photoreceptor 21 is rotatably supported by a main body (not illustrated) of the image forming apparatus 20, and is rotationally driven in the direction of an arrow 23 around a rotation axis 22 by a driving means (not illustrated). The driving means is composed of including, for example, an electric motor and a reduction gear, and by transmitting a driving force to a conductive supporting member constituting a core body of the photoreceptor 21, the photoreceptor 21 is rotationally driven at a predetermined circumferential speed. The charging unit 24, the light exposing means 28, the developing unit 25, the transferring unit 26 and the cleaner 27 are provided in this order along an outer circumferential face of the photoreceptor 21, from an upstream side to a downstream side of the rotational direction of the photoreceptor 21 shown by the arrow 23.

The charging unit 24 is charging means that charges outer circumferential face of the photoreceptor 21 to a predetermined potential. In a present embodiment, the charging unit 24 is implemented by a contact-type charging roller 24a, and a bias power unit 24b that applies voltage on the charging roller 24a.

As the charging means, a charger wire may be used, however, in the charging roller where a high abrasion resistance of the surface of photoreceptor is demanded, the photoreceptor formed with the surface protective layer according to the present invention exerts a greater effect in improvement of the durability.

Therefore, in the image forming apparatus of the present invention, the charging means is preferably a contact charging.

The light exposing means 28 has, for example, a semiconductor laser as a light source, and irradiates between the charging unit 24 and the developing unit 25 of the photoreceptor 21 with a light 28a such as a laser beam outputted from the light source, thereby exposing the charged outer circumferential face of the photoreceptor 21 to light corresponding to the image information. Scanning by the light 28a is repeatedly conducted in the direction of an extension of the rotation axis 22 of the photoreceptor 21, or in the main scanning direction, and the electrostatic latent image is sequentially formed on the surface of the photoreceptor 21 with this scanning.

The developing unit 25 is developing means that develops the electrostatic latent image formed on the surface of the photoreceptor 21 by the light exposure, by a developing agent, and is disposed to face with the photoreceptor 21, and includes a developing roller 25a that supplies the outer circumferential face of the photoreceptor 21 with a toner, and a casing 25b that supports the developing roller 25a so that it is rotatable about a rotation axis which is parallel to the rotation axis 22 of the photoreceptor 21 while storing the developing agent containing the toner in its inner space.

The transferring unit 26 is transferring means that transfers a toner image which is a visible image formed on the outer circumferential face of the photoreceptor 21 by development, onto the transfer paper 30 which is a recording medium supplied between the photoreceptor 21 and the transferring unit 26 from the direction of arrow 29 by means of a conveying means (not illustrated). The transferring unit 26 is, for example, a non-contact type transferring means having the charging means, and transferring the toner image onto the transfer paper 30 by giving a charge having a polarity opposite to that of the toner, to the transfer paper 30.

The cleaner 27 is cleaning means that removes and collects the toner remaining on the outer circumferential face of the photoreceptor 21 after a transferring operation by the transferring unit 26, and includes a cleaning blade 27a that makes the toner remaining on the outer circumferential face of the photoreceptor 21 be peeled off, and a collecting casing 27b for holding the toner peeled off by the cleaning blade 27a. In addition, the cleaner 27 is provided together with an electricity removing lamp (not illustrated).

Further, the image forming apparatus 20 is provided with the fixing unit 31 which is fixing means that fixes a transferred image, on a downstream side where the transfer paper 30 having passed between the photoreceptor 21 and the transferring unit 26 is conveyed. The fixing unit 31 includes a heating roller 31a having a heating means (not illustrated) and a pressurizing roller 31b disposed to be opposite to the heating roller 31a, which forms an abutment part by being pushed by the heating roller 31a.

An image forming operation by the image forming apparatus 20 is conducted in a manner described below. First, as the photoreceptor 21 is rotationally driven in the direction of the arrow 23 by the driving means, the surface of the photoreceptor 21 is uniformly charged at a positive or a negative predetermined potential by means of the charging unit 24 provided on more upstream side of the rotation direction of the photoreceptor 21 than a focal point of the light 28a by the light exposing means 28.

Next, the surface of the photoreceptor 21 is irradiated with the light 28a corresponding to the image information from the light exposing means 28. In the photoreceptor 21, a surface charge in a part irradiated with the light 28a by this light exposures is removed, and a difference arises between the surface potential in the part irradiated with the light 28a, and the surface potential in the part not irradiated with the light 28a, and thus the electrostatic latent image is formed.

The toner is supplied to the surface of the photoreceptor 21 on which the electrostatic latent image is formed, from the developing unit 25 provided on more downstream side of the rotation direction of the photoreceptor 21 than the focal point of the light 28a by the light exposing means 28, and the electrostatic latent image is developed and the toner image is formed.

In synchronization with the light exposure to the photoreceptor 21, the transfer paper 30 is supplied between the photoreceptor 21 and the transferring unit 26. A charge having opposite polarity to the toner is given to the supplied transfer paper 30 by the transferring unit 26, and the toner image formed on the surface of the photoreceptor 21 is transferred onto the transfer paper 30.

The transfer paper 30 onto which the toner image is transferred is conveyed to the fixing unit 31 by the conveying means, and heated and pressurized when it passes through the abutment part between the heating roller 31a and the pressurizing roller 31b of the fixing unit 31, and thus the toner image is fixed on the transfer paper 30 to become a rigid image. The transfer paper 30 on which the image is formed in this manner is discharged outside the image forming apparatus 20 by the conveying means.

On the other hand, also after transferring of the toner image by the transferring unit 26, the toner remaining on the surface of the photoreceptor 21 is peeled off from the surface of the photoreceptor 21 and collected by the cleaner 27. The charge on the surface of the photoreceptor 21 from which the toner is removed in this manner is removed by light from the electricity removing lamp, so that the electrostatic latent image on the surface of the photoreceptor 21 disappears. Thereafter, the photoreceptor 21 is further rotationally driven, and a series of operations starting from charging is repeated again to continuously form images.

Since the image forming apparatus 20 according to the present invention has the photoreceptor 21 having the photosensitive layer in which the triamine compound of the present invention is uniformly dispersed, it is possible to form an image of high quality having no image defect such as black dots.

### EXAMPLES

Hereinafter, the present invention will be described more specifically by way of Production Examples (including comparison), Examples and Comparative Examples, however, the present invention will not be limited to these Production Examples (excluding comparison) and Examples. Here, a chemical structure, a molecular weight, and an elementary analysis of each compound obtained in Production Examples are examined by an apparatus and a condition described below.

### (Chemical Structure)

Nuclear magnetic resonance apparatus: NMR (model: DPX-200, manufactured by Bruker Biospin)

Sample adjustment: about 4 mg sample/0.4 m (CDCl₃)

Measurement mode: ¹H (normal), ¹³C (normal, DPET-135) (Molecular Weight)

Molecular weight measurement apparatus: LC-MS (Finegan LCQ Deca mass spectrometer system, manufactured by Thermo Fisher Scientific K.K.)

LC column: GL-Sciences Inertsil ODS-3 2.1×100 mm

Column temperature: 40° C.

Eluent: methanol:water=90:10

Sample injection amount: 5 μL

Detector: UV254 nm and MS ESI

### (Elementary Analysis)

Elementary analyzing apparatus: Elemental Analysis 2400 manufactured by Perkin Elmer Japan Co., Ltd.

Sample amount: about 2 mg finely weighed

Gas flow rate (mL/min.): He=1.5, O₂=1.1, N₂=4.3

Combustion tube temperatures setting: 925° C.

Reduction tube temperature setting: 640° C.

Herein, elementary analysis was conducted by a carbon (C), hydrogen (H) and nitrogen (N) concurrent quantification method according to a differential thermal conductivity method.

### Production Example 1

Exemplary compound No. 1 was produced according to a reaction formula described below:

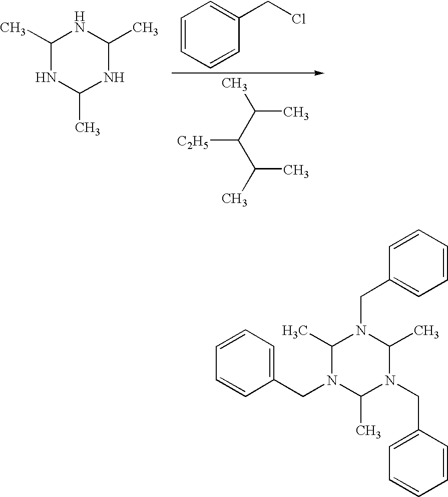

Into 80 mL of anhydrous 1,4-dioxane, 19.4 g (3.05 equivalents) of benzyl chloride and 6.5 g (1.0 equivalent) of trimethyltriazine were added, and ice-cooled in an ice bath. To this solution, 20.2 g (3.1 equivalents) of N,N-diisopropylethylamine was gradually added. Then, a reaction temperature was raised to 100 to 110° C. by gradual heating under stirring, and stirred for 4 hours at the same temperature. After completion of the reaction, this reaction solution was allowed to cool, a generating precipitate was collected by filtration, washed well with water, and allowed to recrystallize in a mixed solvent of ethanol and ethyl acetate (ethanol:ethyl acetate=8:2 to 7:3), to obtain 17.1 g of a white powder compound.

As a result of chemical analysis of the obtained white powder compound, nuclear magnetic resonance apparatus: NMR ¹H-NMR spectrum (normal) showed δ (ppm)=2.2 (d. 9H), 3.1 (q. 3H), 7.1 to 7.8 (m. 15H).

In addition, ¹³C-NMR spectrum (normal, DEPT-135) showed δc (ppm)=40.8 (CH₃, signal intensity 3), 77.4 (CH, signal intensity 3), 58.1 (CH₂, signal intensity 3), 127.0 (CH, signal intensity 3), 128.5 (CH, signal intensity 6), 128.9 (CH, signal intensity 6), 139.4 (C, signal intensity 3).

Further, in the molecular weight measuring apparatus: LC-MS, a peak was observed at 400.5 which corresponds to a molecular ion [M+H]⁺ in which a proton is added to the Exemplary compound No. 1 (calculated value of molecular weight: 399.3).

In addition, Elementary analysis values of the white powder compound were as follows:

### <Elementary Analysis Values of Exemplary Compound No. 1>

Theoretical values C: 81.16%, H: 8.32%, N: 10.52%

Actual values C: 80.84%, H: 7.98%, N: 10.24%

Analysis results of the NMR, the LC-MS, elementary analysis and the like proved that the obtained white powder compound was a triamine compound of the Exemplary compound No. 1 (yield: 85.0%). Further, analysis results of HPLC in measurement of the LC-MS proved that a purity of the obtained Exemplary compound No. 1 was 98.8%.

### Production Examples 2 to 8

In Production Example 1, Exemplary compounds No. 3, 7, 9, 23, 28, 34 and 37 were produced by conducting completely the same operation using respective material compounds shown in Table described below as the amine compound represented by the General formula (6) and as the halogen compound represented by General formula (8). Here, in Table described below, material compounds of the Exemplary compound No. 1 are also shown.

TABLE 2

| Com- | | | | |
|---|---|---|---|---|
| pound | Triamine compound (6) | Halogen compound (8) | | |
| ProductionExample 1ExemplarycompoundNo. 1 | 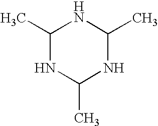 | 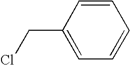 | 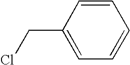 | 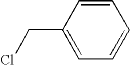 |
| ProductionExample 2ExemplarycompoundNo. 3 | 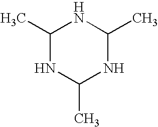 | 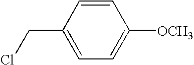 | 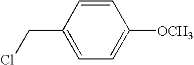 | 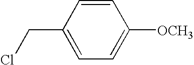 |
| ProductionExample 3ExemplarycompoundNo. 7 | 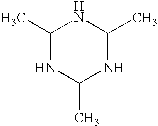 | 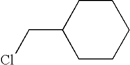 | 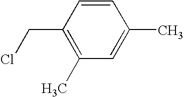 | 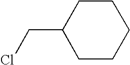 |
| ProductionExample 4ExemplarycompoundNo. 9 | 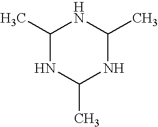 | 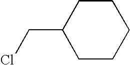 | 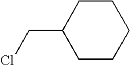 | 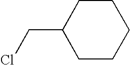 |
| ProductionExample 5ExemplarycompoundNo. 23 | 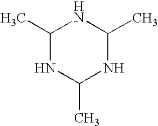 | 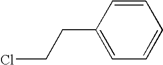 | 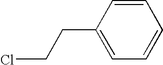 | 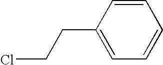 |
| ProductionExample 6ExemplarycompoundNo. 28 | 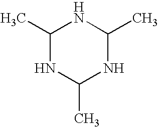 | 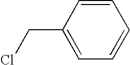 | 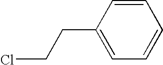 | 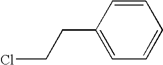 |
| ProductionExample 7ExemplarycompoundNo. 34 | 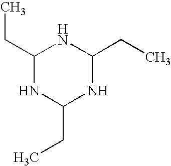 | 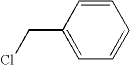 | 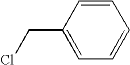 | 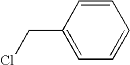 |
| ProductionExample 8ExemplarycompoundNo. 37 | 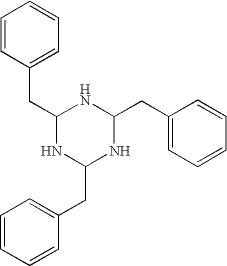 | 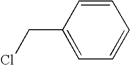 | 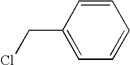 | 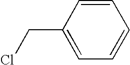 |

Also the elementary analysis values as well as calculated values and actual values [M+H] by the LC-MS of molecular weight of respective Exemplary compounds obtained in the above Production Examples 1 to 8 are shown in Table described below.

TABLE 3

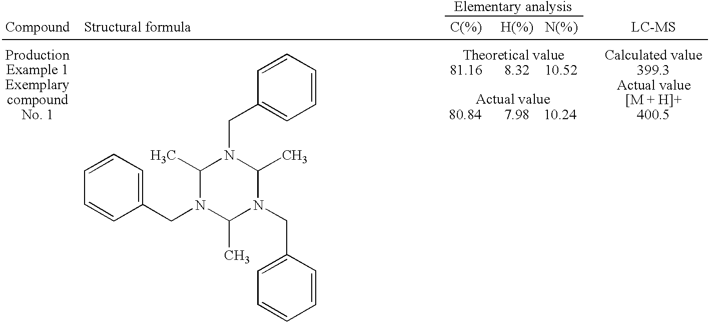

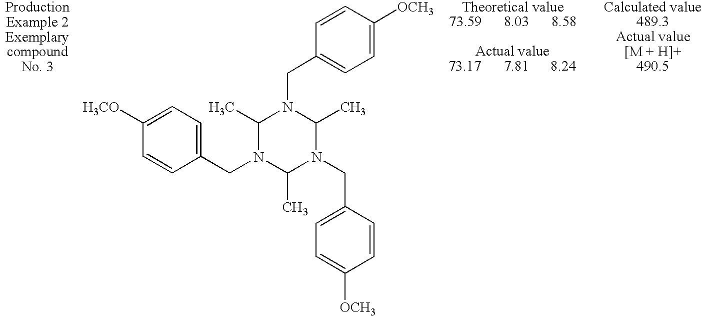

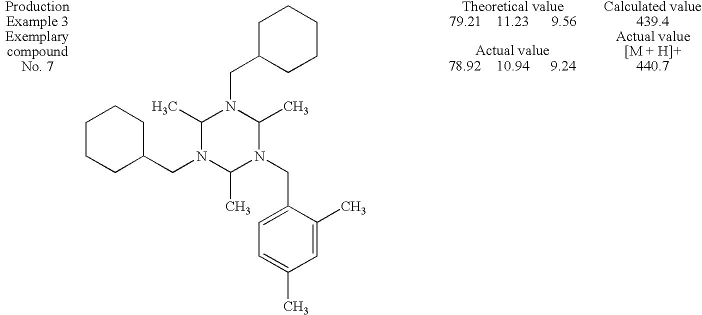

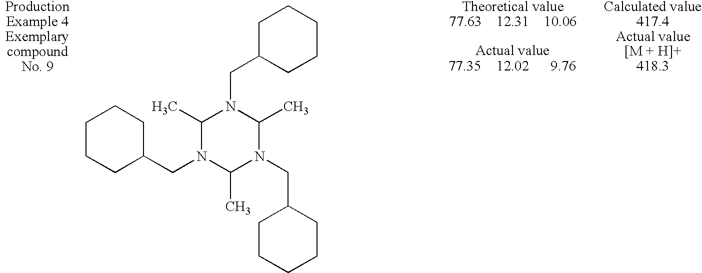

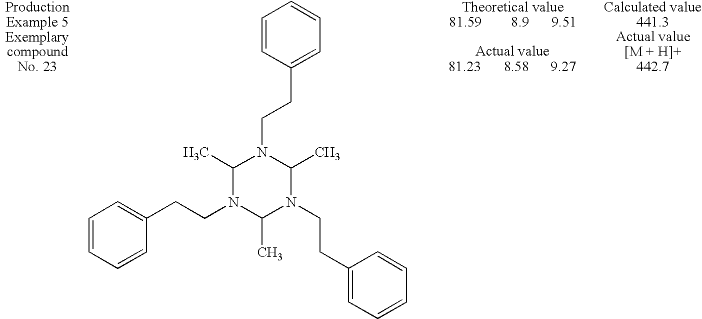

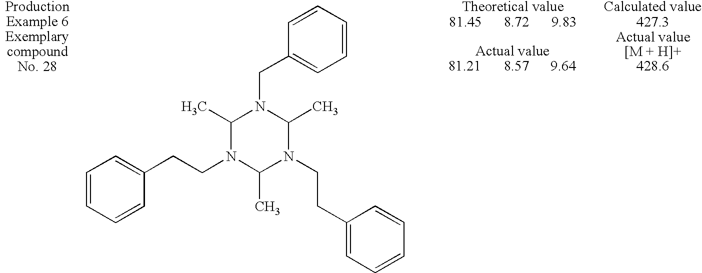

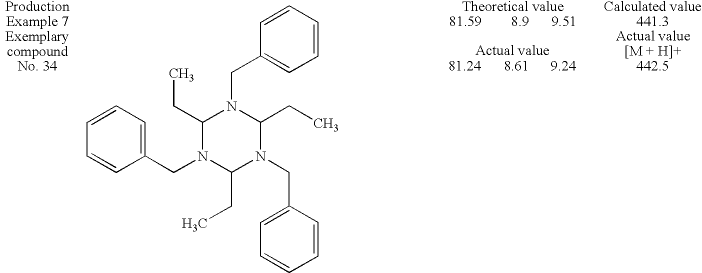

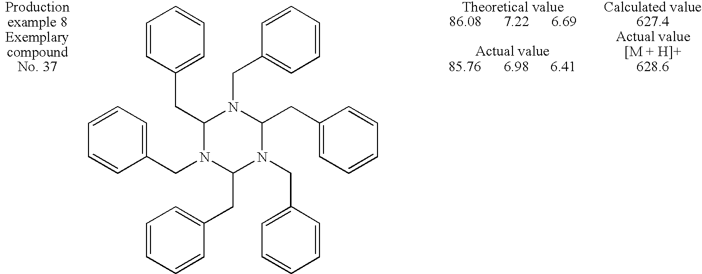

### Production Example 9

Production Example by synthesis of a comparative diamine compound and a reaction treatment in aqueous system using an inorganic base

According to a synthesis example described in Japanese Patent Application Laid-open Publication No. 5-158258, 6.2 g of a diamine compound represented by a chemical structural formula (9) described below which is a described Exemplary compound (No. 10) (hereinafter, referred to as a “diamine compound synthesized by Comparative Production Example”) was obtained

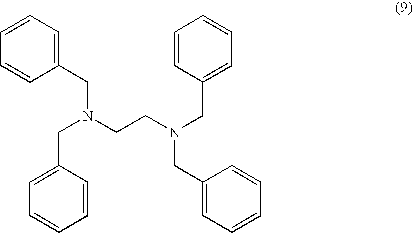

### “Diamine Compound Synthesized by Comparative Production Example” (9)

Elementary analysis values of the obtained diamine compound synthesized by Comparative Production Example are as follows.

### <Elementary Analysis Values of Diamine Compound Synthesized by Comparative Production Example>

Theoretical values C: 85.67%, H: 7.67%, N: 6.66%

Actual values C: 84.21%, H: 6.38%, N: 5.87%

Here, the obtained diamine compound synthesized by Comparative Production Example was analyzed by the LC-MS, and a peak was observed at 421.5 which corresponds to the molecular ion [M+H]⁺ in which the proton is added to an objective compound represented by the above chemical structural formula (calculated value of molecular weight: 420.26).

The analysis results of the elementary analysis and the LC-MS proved that the obtained compound was the diamine compound of the Exemplary compound (No. 10) described in Japanese Patent Application Laid-open Publication No. 5-158258. Also the analysis result of the HPLC in measurement of the LC-MS demonstrated that a purity of the obtained diamine compound was 96.3%.

From these results, it was confirmed that a compound having an object structure was obtained because a molecular ion peak of the objective substance was observed by the LC-MS. However, from the elemental analysis values and the analysis results of the HPLC, it was suggested that the obtained compound contained impurities. This, however, would be caused by a matter other than our synthetic method.

### Example 1

As described below, a photoreceptor containing the Exemplary compound No. 1 which is the triamine compound according to the present invention produced in Production Example 1, in the charge transporting layer was fabricated. As the conductive supporting member, an aluminum tube having a diameter of 30 mm, a total length of 340 mm, and a thickness of 1 mm was used.

7 parts by weight of titanium oxide (trade name: TIPAQUE TTO55A, manufactured by ISHIHARA SANGYO KAISYA LTD.) and 13 parts by weight of a copolymer nylon resin (trade name: Amilan CM8000, manufactured by TORAY INDUSTRIES, INC.) were added into a mixed solvent of 159 parts by weight of methyl alcohol and 106 parts by weight of 1,3-dioxylane, and dispersed for 8 hours by a paint shaker, to prepare 10 kg of an application liquid for forming the intermediate layer. This application liquid for forming the intermediate layer was applied on an aluminum surface of the aluminum tube which is the conductive supporting member by the dip coating method, and naturally dried, to form an intermediate layer having a film thickness of 1 μm.

Then, 1 part by weight of X-type nonmetallic phthalocyanine (FastogenBlue 8120, manufactured by DIC corporation) and 1 part by weight of a butyral resin (trade name: #6000-C, manufactured by DENKI KAGAKU KOGYO KABUSHIKI KAISYA) were mixed with 98 parts by weight of methylethyl ketone, and dispersed by a paint shaker, to prepare 5 kg of an application liquid for forming the charge generating layer. This application liquid for forming the charge generating layer was applied on the surface of the intermediate layer formed previously, by a similar manner as is a case of the intermediate layer, and naturally dried to form a charge generating layer having a film thickness of 0.4 μm.

Then 2.5 parts by weight of the triamine compound of the Exemplary compound No. 1 produced in Production Example 1, 100 parts by weight of the charge transporting material represented by Structural formula (10) described below, and 180 parts by weight of a polycarbonate resin (trade name: Iupilon Z400, manufactured by Mitsubishi Gas Chemical Company Inc.) were mixed, and 10 kg of an application liquid for forming the charge transporting layer having solid content of 10% in toluene as a solvent was prepared. This application liquid for forming the charge transporting layer was applied in a similar manner as is the case of the intermediate layer on a surface of the previously formed charge generating layer so that two kinds of charge transporting layers were formed having different film thickness, 15 μm and 28 μm, and dried at 130° C. for an hour, to fabricate the laminate type photoreceptor of the present invention having the laminate structure including the intermediate layer, the charge generating layer and the charge transporting layer sequentially laminated on the conductive supporting member similarly to the photoreceptor 17 shown in FIG. 7.

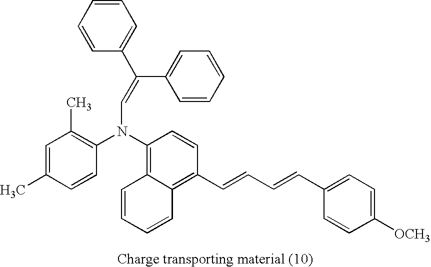

### Examples 2 to 4

The laminate type photoreceptor of the present invention having the laminate structure including the intermediate layer, the charge generating layer and the charge transporting layer sequentially laminated on the conductive supporting member were fabricated in a similar manner as Example 1 except that the Exemplary compounds No. 3, 9 and 37 were respectively used in place of the Exemplary compound No. 1 which is a triamine compound of the present invention.

### Example 5

The laminate type photoreceptor of the present invention having the laminate structure including the intermediate layer, the charge generating layer and the charge transporting layer sequentially laminated on the conductive supporting member was fabricated in a similar manner as Example 1 except that a compound represented by Structural formula (11) described below was used as the charge transporting material.

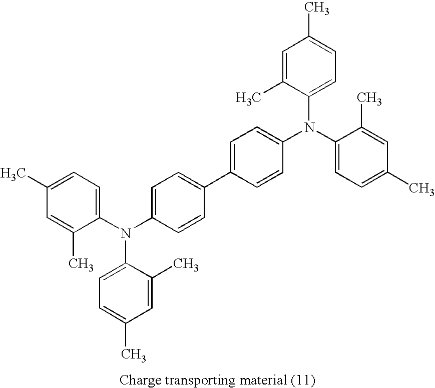

### Example 6

The laminate type photoreceptor of the present invention having the laminate structure including the intermediate layer, the charge generating layer and the charge transporting layer sequentially laminated on the conductive supporting member was fabricated in a similar manner as Example 1 except that a compound represented by Structural formula (12) described below was used as the charge transporting material.

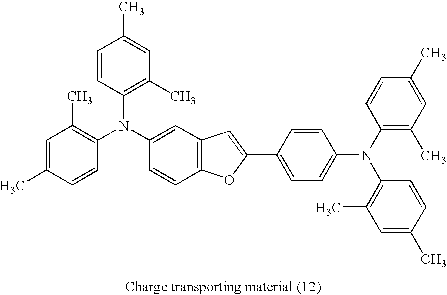

### Example 7

The laminate type photoreceptor of the present invention having the laminate structure including the intermediate layer, the charge generating layer and the charge transporting layer sequentially laminated on the conductive supporting member was fabricated in a similar manner as Example 1 except that 0.1 parts by weight of the triamine compound of the Exemplary compound No. 1 was used.

### Example 8

The laminate type photoreceptor of the present invention having the laminate structure including the intermediate layer, the charge generating layer and the charge transporting layer sequentially laminated on the conductive supporting member was fabricated in a similar manner as Example 1 except that 20 parts by weight of the triamine compound of the Exemplary compound No. 1 was used.

### Example 9

### Fabrication of Monolayer Type Photoreceptor Containing Triamine Compound in Monolayer Type Photosensitive Layer

1 part by weight of X-type nonmetallic phthalocyanine, 12 parts by weight of the polycarbonate resin (trade name: Iupilon Z400, manufactured by Mitsubishi Gas Chemical Company Inc.), 0.25 part by weight of the triamine compound of the Exemplary compound No. 1, 10 parts by weight of the charge transporting material represented by the Structural formula (10), 5 parts by weight of 3,5-dimethyl-3′,5′-di-t-butyldiphenoquinone, 0.5 parts by weight of 2,6-di-t-butyl-4-methylphenol and 65 parts by weight of THF were dispersed for 12 hours by the ball mill, to prepare 5 kg of an application liquid for forming the monolayer type photosensitive layer.

Then on the intermediate layer provided in a similar manner as in Example 1, the application liquid was applied, dried at 130° C. for 1 hours, to form two kinds of the photosensitive layers having different film thicknesses of the photosensitive layer of 15 μm and 28 μm, respectively. In this manner, a monolayer type photoreceptor having the configuration as shown in FIG. 3 was fabricated.

### Example 10

### Fabrication of Monolayer Type Photoreceptor Containing Triamine Compound Both in Monolayer Type Photosensitive Layer and Surface Protective Layer Provided on Photosensitive Layer

1.8 parts by weight of the polycarbonate resin (trade name: Iupilon Z400, manufactured by Mitsubishi Gas Chemical Company Inc.), and 1.8 parts by weight of silica (TS-610: manufactured by Cabot Specialty Chemicals Inc.) were mixed with 32.4 parts by weight of tetrahydrofuran. A mixture obtained was dispersed for 5 hours by the ball mill using ZrO₂ beads (φ3 mm) as media, to prepare 5 kg of a primary dispersed application liquid for the surface protective layer. Then 2.5 parts by weight of the triamine compound of the Exemplary compound No. 1, 100 parts by weight of the charge transporting material represented by the Structural formula (10), 139.8 parts by weight of the polycarbonate resin (trade name: Iupilon Z400, manufactured by Mitsubishi Gas Chemical Company Inc.), and 5 parts by weight of an antioxidant (SUMILIZER BHT: manufactured by Sumitomo Chemical Co., Ltd.) were mixed with 984 parts by weight of the tetrahydrofuran, and dissolved. To this solution, 3.6 parts by weight of the primary dispersed application liquid for a surface protective layer was mixed, and stirred for 15 hours, to prepare 10 kg of a secondary dispersed application liquid for the surface protective layer.

This application liquid for the surface protective layer was applied on the surface of the previously provided photosensitive layer by the spraying method, to form a surface protective layer to have a film thickness of 1 μm after drying.

In this manner, a monolayer type photoreceptor having configuration as shown in FIG. 4 was fabricated.

### Example 11

### Fabrication of Monolayer Type Photoreceptor Containing Triamine Compound Only in Surface Protective Layer Formed on Monolayer Type Photosensitive Layer

A monolayer type photoreceptor having the configuration shown in FIG. 4 was fabricated in a similar manner as in the Example 10 except that the triamine compound of Exemplary compound No. 1 was not used in the photosensitive layer in Example 10.

### Example 12

### Fabrication of Laminate Type Photoreceptor Containing Triamine Compound Both in Charge Transporting Layer of Laminate Type Photosensitive Layer and Surface Protective Layer Formed on Photosensitive Layer

1.8 parts by weight of the polycarbonate resin (trade name: Iupilon Z400, manufactured by Mitsubishi Gas Chemical Company Inc.) and 1.8 parts by weight of the silica (TS-610: manufactured by Cabot Specialty Chemicals Inc.) were mixed with 32.4 parts by weight of the tetrahydrofuran. A mixture obtained was dispersed for 5 hours by the ball mill using ZrO₂ beads (φ3 mm) as media, to prepare 5 kg of a primary dispersed application liquid for the surface protective layer. Then, 2.5 parts by weight of the triamine compound of the Exemplary compound No. 1, 100 parts by weight of the charge transporting material represented by the Structural formula (10), 139.8 parts by weight of the polycarbonate resin (trade name: Iupilon Z400, manufactured by Mitsubishi Gas Chemical Company Inc.), and 5 parts by weight of the antioxidant (SUMILIZER BHT: manufactured by Sumitomo Chemical Co., Ltd.) were mixed with 984 parts by weight of the tetrahydrofuran and dissolved. This solution was mixed with 3.6 parts by weight of the primary dispersed application liquid for the surface protective layer, and stirred for 15 hours, to prepare 10 kg of a secondary dispersed application liquid for the surface protective layer.

This application liquid for forming the surface protective layer was applied on the surface of the photoreceptor fabricated in Example 1 by the spraying method so that a film thickness after drying was 1 μm, to form a surface protective layer.

In this manner, a laminate type photoreceptor having the configuration as shown in FIG. 8 was fabricated.

### Example 13

### Fabrication of Laminate Type Photoreceptor Containing Triamine Compound Only in Surface Protective Layer Provided on Laminate Type Photosensitive Layer

In Example 12, a laminate type photoreceptor having the configuration as shown in FIG. 8 was fabricated in a similar manner as in Example 12 except that the triamine compound of the Exemplary compound No. 1 was not used in the charge transporting layer.

### Comparative Example 1

A laminate type photoreceptor was fabricated in a similar manner as in Example 1, except that the triamine compound according to the present invention was not used.

### Comparative Example 2

A laminate type photoreceptor was fabricated in a similar manner as in Example 5, except that the triamine compound according to the present invention was not used.

### Comparative Example 3

A laminate type photoreceptor was fabricated in a similar manner as in Example 6, except that the triamine compound according to the present invention was not used.

### Comparative Example 4

A laminate type photoreceptor was fabricated in a similar manner as in Example 1, except that hydroxyethyldibenzyl amine (compound described in Japanese Patent Application Laid-open Publication No. 3-172852) was used in place of the triamine compound of the present invention.

### Comparative Example 5

A laminate type photoreceptor was fabricated in a similar manner as in Example 1, except that a triamine compound synthesized in Comparative Production Example (Production example 9) (compound described in Japanese Patent Application Laid-open Publication No. 5-158258) was used in place of the triamine compound of the present invention.

For each photoreceptor of Examples 1 to 8 and Comparative examples 1 to 5 fabricated in the manner as described above, (a) ozone gas resistance and (b) stability of electric characteristic were evaluated in a manner described below, and further a general judgment of (c) photoreceptor performance was conducted.

(a) ozone gas resistance

### [Evaluation by Evaluator]

Each photoreceptor for evaluation by evaluator of Examples 1 to 8 and Comparative Examples 1 to 5 (layer thickness of charge transporting layer: 15 μm) was installed in a testing copying machine, a surface potential V₁ (V) of the photoreceptor directly after charging and a surface potential V₂ (V) of the photoreceptor after elapse of 3 seconds from charging were measured under N/N (Normal Temperature/Normal Humidity) environment of temperature of 25° C. and relative humidity of 50%. As the testing copying machine, a commercially available copying machine AR-F 330 (trade name, manufactured by SHARP Corporation) having a corona discharger/charger as the charging means of the photoreceptor, provided with a surface potential meter (trade name: CATE751, manufactured by Gen-Tech, Inc.) so as to allow a measurement of the surface potential of the photoreceptor in a course of the image formation was used. The surface potential V₁ (V) of the photoreceptor directly after charging and the surface potential V₂ (V) of the photoreceptor after elapse of 3 seconds from charging were assigned to General formula (1) described below, and a charge retaintivity DD (%) was calculated, which was referred to as an initial charge retaintivity DDₒ.

Charge retaintivity DD(%)=V₂(V)/V₁(V)×100 (1)

Then, using an ozone generation and control apparatus (trade name: OES-10A, manufactured by Dylec Inc.), each photoreceptor was exposed to ozone for 20 hours in a hermetically-sealed container in which ozone concentration was adjusted to about 7.5 ppm (examined by an ozone concentration meter MODEL 1200 (trade name), manufactured by Dylec Inc.). After exposure to ozone, each photoreceptor was left still for two hours in the N/N environment of 25° C. of temperature and 50% of relative humidity, and then the charge retaintivity DD (%) was determined likewise as before ozone exposure, which was referred to as a charge retaintivity after ozone exposure DD_{O2}.

A value obtained by subtracting the charge retaintivity after ozone exposure DD_{O2} from a charge retaintivity before ozone exposure, or the initial charge retaintivity DDₒ was determined as a variation in charge retaintivity ΔDD (=DD_{O}−DD_{O2}), which was regarded as an evaluation index of an ozone gas resistance.

Here, in Examples 9 to 13, a charging unit of the copying machine was modified so that the measurement was conducted by positive charge.

### [Evaluation by an Actual Machine]

Each photoreceptor for actual machine evaluation (layer thickness of charge transporting layer: 28 μm) of Examples 1 to 8 and Comparative Examples 1 to 5 was installed in the commercially available copying machine AR-F330 (trade name, manufactured by SHARP Corporation) having a corona discharger/charger as the charging means of the photoreceptor, and a test image of a predetermined pattern was actually printed on fifty thousand sheets of the recording paper in the N/N environment of 25° C. of temperature and 50% of relative humidity. After stopping operation of the copying machine for an hour from a time point when actual printing of fifty thousand sheets completed, a halftone image was copied on the recording paper, which was provided as a first image for evaluation. Then the test image of the predetermined pattern was actually printed again on fifty thousand sheets of the recording paper in the N/N environment of 25° C. of temperature and 50% of relative humidity, and after stopping operation of the copying machine for an hour from the time point when actual printing of fifty thousand sheets completed, the halftone image was copied on the recording paper, which was provided as a second image for evaluation.

Here, in Examples 9 to 13, measurement was conducted while adapting each of charging, developing, transferring, charge removing units of the copying machine to positive charge, and changing a toner into a positive chargeable toner.

The formed first image for evaluation and the second image for evaluation were respectively observed visually, and an image quality in a site of the recording paper corresponding to the part where the toner image was transferred from the site of the photoreceptor that was arranged near the corona discharger/charger at the time when operation of copying machine was stopped was evaluated according to a degree of an occurrence of the image defect such as a pin hole or a black band, which was referred to as an evaluation index of the ozone gas resistance. Evaluation criteria of the image quality are as follows.

\textcircled{•}: excellent (no image defect occurs in both of the first image for evaluation and the second image for evaluation)

◯: good (slight but negligible image defect occurs in either one or both of the first image for evaluation and the second image for evaluation)

Δ: approved (slight but practically unproblematic image defect occurs in either one or both of the first image for evaluation and the second image for evaluation)

x: disapproved (significant image defect occurs in either one or both of the first image for evaluation and the second image for evaluation, so that practical use is not allowed)

Taking the above variation in charge retaintivity ADD and evaluation result of the image quality together, the ozone gas resistance of the photoreceptor was evaluated. Evaluation criteria of the ozone gas resistance are as follows.

\textcircled{•}: excellent (ΔDD is less than 3.0% and image quality is excellent (\textcircled{•}))

◯: good (ADD is 3.0% or more and less than 7.0% and image quality is excellent (\textcircled{•}), or ΔDD is less than 7.0% and image quality is good (◯))

Δ: practically unproblematic (ΔDD is less than 7.0% and image quality is approved (Δ))

x: poor (ADD is 7.0% or more or image quality is disapproved (x))

### (b) Stability of Electric Characteristic

Each photoreceptor for the actual machine evaluation (layer thickness of charge transporting layer: 28 μm) of Examples 1 to 8 and Comparative Examples 1 to 5 was installed in the testing copying machine, and the stability of the electric characteristic was evaluated in a manner described below in each of L/L (Low Temperature/Low Humidity) environment of 5° C. of temperature and 20% of relative humidity, and H/H (High Temperature/High Humidity) environment of temperature of 35° C. of temperature and 85% of relative humidity. As the testing copying machine, the commercially available copying machine AR-F 330 (trade name, manufactured by SHARP Corporation) having (trade name, manufactured by SHARP Corporation) corona discharger/charger as the charging means of the photoreceptor, provided with the surface potential meter (trade name: manufactured by CATE751, Gen-Tech, Inc.) so as to allow measurement of the surface potential of the photoreceptor in the course of the image formation was used. The copying machine AR-F330 is an image forming apparatus of a negative charge type which conducts an electrophotographic process while charging the surface of the photoreceptor negatively.

Using the testing copying machine equipped with each photoreceptor of Examples 1 to 8 and Comparative Examples 1 to 5, a surface potential of the photoreceptor directly after charging operation by the charger was measured as V0 (V), which was referred to as an initial charge potential V0₁ (V). Also, a surface potential of the photoreceptor directly after light exposure by laser was measured as a residual potential Vr (V), which was referred to as an initial residual potential Vr₁.

Then after copying the test image of the predetermined pattern onto three hundred thousand sheets of the recording paper successively, the charge potential V0 and the residual potential Vr were measured in a similar manner as the initial ones, which were referred to as a charge voltage V0₂ after the repeated usage and a residual potential Vr₂ after the repeated usage. An absolute value of a difference between an initial charging potential V0₁ and the charge potential V0₂ after the repeated usage was determined as a variation in charge potential ΔV0 (=|V0₁−V0₂|). Further, an absolute value of a difference between an initial residual potential Vr₁ and the residual potential Vr₂ after the repeated usage was determined as a variation in residual potential ΔVr (=|Vr₁−Vr₂|). The stability of the electric characteristic was evaluated according to the variation in charge potential ΔV0 and the variation in residual potential ΔVr as evaluation indexes.

Evaluation criteria of the stability of the electric characteristic in L/L environment are as follows:

\textcircled{•}: excellent (ΔV0 is 35V or less and ΔVr is 55V or less)

◯: good (ΔV0 is 35V or less and ΔVr is more than 55V and 80V or less, or ΔV0 is more than 35V and 75V or less and ΔVr is 55V or less)

Δ: practically unproblematic (ΔV0 is more than 35V and 75V or less, and ΔVr is more than 55V and 80V or less)

x: poor (ΔV0 is more than 75V, or ΔVr is more than 80V)

Evaluation criteria of the stability of the electric characteristic in H/H environment are as follows:

\textcircled{•}: excellent (ΔV0 is 15V or less and ΔVr is 105V or less)

◯: good (ΔV0 is 15V or less and ΔVr is more than 105V and 125V or less, or ΔV0 is more than 15V and 30V or less and ΔVr is 105V or less)

Δ: practically unproblematic (ΔV0 is more than 15V and 30V or less, and ΔVr is more than 105V and 125V or less)

x: poor (ΔV0 is more than 30V, or ΔVr is more than 125V)

Taking evaluation result in L/L environment and evaluation result in H/H environment together, general evaluation of the stability of the electric characteristic was made. Evaluation criteria of the general evaluation of the stability of the electric characteristic are as follows.

\textcircled{•}: excellent (excellent (\textcircled{•}) in both L/L environment and H/H environment)

◯: good (good (◯) in either one of L/L environment and H/H environment and excellent (\textcircled{•}) or good (◯) in the other of L/L environment and H/H environment)

Δ: practical unproblematic (practically unproblematic (Δ) in either one of L/L environment and H/H environment, and not poor (x) in the other of L/L environment and H/H environment)

x: poor (poor (X) in either one or both of L/L environment and H/H environment)

### (c) General Judgment of Photoreceptor Performance

Taking evaluation result of the ozone gas resistance and general evaluation result of the stability of the electric characteristic together, the general judgment of a photoreceptor performance was made. Evaluation criteria of the general judgment are as follows:

\textcircled{•}: excellent (excellent (\textcircled{•}) both in ozone gas resistance and stability of electric characteristic)

◯: good (good (◯) in either of ozone gas resistance and stability of electric characteristic, and excellent (\textcircled{•}) or good (◯) in the other of ozone gas resistance and stability of electric characteristic)

Δ: practically unproblematic (practically unproblematic (Δ) in either one of ozone gas resistance and stability of electric characteristic, and not poor (x) in the other of ozone gas resistance and stability of electric characteristic)

x: poor (poor (x) in either one or both of ozone gas resistance and stability of electric characteristic)

These evaluation results are shown in Table 4.

TABLE 4

| | | | Charge | | | | |
|---|---|---|---|---|---|---|---|
| | | Triamine compound | transporting | Evaluation of gas resistance characteristic | | | |
| | Triamine compound, | Exemplary compound | material compound | Initial charge | Variation in charge | Image | |
| Example | Exemplary compound No. | adding amount (%) | No. | retaintivity (DD) | retaintivity ΔDD | quality | Evaluation |
| 1 | 1 | 2.5 | 10 | 91.3 | 2.7 | ⊚ | ⊚ |
| 2 | 3 | 2.5 | 10 | 91.2 | 2.9 | ⊚ | ⊚ |
| 3 | 9 | 2.5 | 10 | 90.6 | 3.1 | ⊚ | ◯ |
| 4 | 37 | 2.5 | 10 | 90.5 | 2.8 | ⊚ | ⊚ |
| 5 | 1 | 2.5 | 11 | 91.3 | 2.8 | ⊚ | ⊚ |
| 6 | 1 | 2.5 | 12 | 91.5 | 3.1 | ⊚ | ◯ |
| 7 | 1 | 0.1 | 10 | 91.4 | 3.1 | ⊚ | ◯ |
| 8 | 1 | 2.0 | 10 | 91.7 | 3.0 | ⊚ | ◯ |
| 9 | 1 | 2.5 | 10 | 87.2 | 3.7 | ⊚ | ◯ |
| 10 | 1 | 2.5/2.5 | 10 | 88.5 | 3.5 | ⊚ | ◯ |
| 11 | 1 | 2.5/0 | 10 | 87.7 | 3.9 | ⊚ | ◯ |
| 12 | 1 | 2.5/2.5 | 10 | 92.5 | 2.8 | ⊚ | ⊚ |
| 13 | 1 | 2.5/0 | 10 | 90.5 | 3.2 | ⊚ | ◯ |
| Comparative | — | — | 10 | 90.2 | 21.5 | X | X |
| example 1 | | | | | | | |
| Comparative | — | — | 11 | 91.8 | 22.6 | X | X |
| example 2 | | | | | | | |
| Comparative | — | — | 12 | 90.3 | 21.7 | X | X |
| example 3 | | | | | | | |
| Comparative | Hydroxyethyl | 2.5 | 8 | 90.5 | 4.2 | Δ | Δ |
| example 4 | dibenzylamine | | | | | | |
| | (compound described in | | | | | | |
| | Japanese Patent | | | | | | |
| | Application Laid-open | | | | | | |
| | Publication No. 3-172852) | | | | | | |
| Comparative | Diamine compound (9) | 2.5 | 8 | 90.5 | 4.2 | Δ | Δ |
| example 5 | (compound described in | | | | | | |
| | Japanese Patent | | | | | | |
| | Application Laid-open | | | | | | |
| | Publication No. 5-158258) | | | | | | |

| | Electric characteristic after repeated usage | | | | | | | | | | | |
|---|---|---|---|---|---|---|---|---|---|---|---|---|
| | L/L potential characteristic | | | | | H/H potential characteristic | | | | | General | General |
| Example | Vo | Δ Vo | Vr | Δ Vr | Evaluation | Vo | Δ Vo | Vr | Δ Vr | Evaluation | evaluation | judgment |
| 1 | −667 | 30 | −51 | 37 | ⊚ | −660 | 14 | −31 | −54 | ⊚ | ⊚ | ⊚ |
| 2 | −672 | 32 | −61 | 41 | ⊚ | −664 | 15 | −27 | −67 | ⊚ | ⊚ | ⊚ |
| 3 | −673 | 28 | −52 | 43 | ⊚ | −671 | 16 | −32 | −65 | ◯ | ◯ | ◯ |
| 4 | −672 | 31 | −50 | 39 | ⊚ | −661 | 21 | −28 | −67 | ◯ | ◯ | ◯ |
| 5 | −673 | 29 | −61 | 44 | ⊚ | −667 | 18 | −32 | −51 | ◯ | ◯ | ◯ |
| 6 | −672 | 33 | −41 | 38 | ⊚ | −665 | 20 | −32 | −53 | ◯ | ◯ | ◯ |
| 7 | −665 | 31 | −43 | 42 | ⊚ | −661 | 22 | −33 | −54 | ◯ | ◯ | ◯ |
| 8 | −678 | 28 | −51 | 41 | ⊚ | −665 | 13 | −35 | −57 | ⊚ | ⊚ | ⊚ |
| 9 | 670 | 45 | 55 | 60 | ◯ | 645 | 25 | 50 | −70 | ◯ | ◯ | ◯ |
| 10 | 675 | 50 | 65 | 70 | ◯ | 655 | 23 | 55 | −75 | ◯ | ◯ | ◯ |
| 11 | 674 | 43 | 60 | 75 | ◯ | 650 | 25 | 50 | −65 | ◯ | ◯ | ◯ |
| 12 | −677 | 31 | −50 | 52 | ⊚ | −665 | 14 | −34 | −56 | ⊚ | ⊚ | ⊚ |
| 13 | −673 | 30 | −55 | 47 | ⊚ | −663 | 13 | −30 | −69 | ⊚ | ⊚ | ⊚ |
| Comparative | −655 | 29 | −52 | 48 | ⊚ | 655 | 16 | −33 | 65 | ⊚ | ⊚ | X |
| example 1 | | | | | | | | | | | | |
| Comparative | −683 | 30 | −54 | 42 | ⊚ | 649 | 17 | −31 | 67 | ⊚ | ⊚ | X |
| example 2 | | | | | | | | | | | | |
| Comparative | −665 | 33 | −48 | 47 | ⊚ | 642 | 14 | −35 | 62 | ⊚ | ⊚ | X |
| example 3 | | | | | | | | | | | | |
| Comparative | −648 | 41 | −63 | 53 | ◯ | −645 | 9 | −43 | 110 | ◯ | ◯ | Δ |
| example 4 | | | | | | | | | | | | |
| Comparative | −648 | 41 | −63 | 53 | ◯ | −645 | 9 | −43 | 110 | ◯ | ◯ | Δ |
| example 5 | | | | | | | | | | | | |

From a comparison between Examples 1 to 6 and Comparative Examples 1 to 3, it was found that the photoconductors of Examples 1 to 6 containing the triamine compound of the present invention are more excellent in the ozone gas resistance and the stability of the electric characteristic, and show the excellent electric characteristic even after the repeated usage, compared to the photoconductors of Comparative Example 1 to 3.

It is also found that a uniform performance is also exhibited for charge transporting materials having different backbones, and a range of application for various charge transporting materials is wide.

From Examples 7 and 8, it can be found that an excellent effect is exhibited when the amount of the triamine compound of the present invention added is in the range of 0.1 to 20 parts by weight, relative to 100 parts by weight of the charge transporting material.

From a comparison between Example 1 and Comparative Examples 4 and 5, as for the known amine-based or diamine-based materials proposed in similar purposes of the present invention, a difference in effect clearly appears when evaluation is made considering the image quality, and it can be found that the photoreceptor of Example 1 using the triamine-based compound of the present invention is superior.

Further, although the diamine-based materials are within acceptable range in terms of the image quality compared to the triamine-based compound of the present invention, however, they have problematic because deterioration in the aspect of the electric characteristic after the repeated usage is significant due to an influence of inorganic metallic impurities which is attributable to the production method.

A comparison between Example 1 and Comparative example 5 reveals a difference in a characteristic aspect between similar amine compounds remarkably appears in the aspect of the electric characteristic after the repeated usage.

As described above, by containing the triamine compound of the present invention represented by the General formula (1) in the photoreceptor, it is possible to provide a photoreceptor having the excellent electric characteristic such as a chargeability, and a responsibility as well as the excellent ozone gas resistance, and the excellent characteristic stability such that the excellent electric characteristics are not deteriorated even after the repeated usage.

### INDUSTRIAL APPLICABILITY

The triamine compound of the present invention provides the excellent ozone resistance and causes no adverse effect in the aspect of the electrophotographic characteristic by being contained in the photosensitive layer containing the organic photoconductive material, and hence is preferable as a compound used together with an organic photoconductive material.

1. An electrophotographic photoreceptor, which is the electrophotographic photoreceptor including a monolayer type photosensitive layer containing a charge generating material and a charge transporting material and an optional surface protective layer laminated on a conductive supporting member made of a conductive material, or which is the electrophotographic photoreceptor including a laminate type photosensitive layer having a charge generating layer containing the charge generating material and a charge transporting layer containing the charge transporting material laminated in this order, and the optional surface protective layer laminated on the conductive supporting member made of the conductive material, wherein
i) when the surface protective layer is not formed on a surface of the monolayer type photosensitive layer or the laminate type photosensitive layer, the charge transporting layer of the monolayer type photosensitive layer or the laminate type photosensitive layer contains a triamine compound represented by General formula (1) described below;
ii) when the surface protective layer is formed on respective surface of the monolayer type photosensitive layer or the laminate type photosensitive layer, both of the monolayer type photosensitive layer and the surface protective layer, or both of the charge transporting layer of the laminate type photosensitive layer and the surface protective layer contain a triamine compound represented by General formula (1); or
the monolayer type photosensitive layer or the surface protective layer of the laminate type photosensitive layer contains the triamine compound represented by General formula (1) described below: 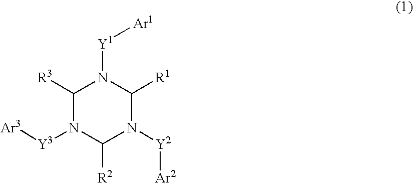
wherein, Ar¹, Ar² and Ar³, which are the same or different with each other, represent an optionally substituted aryl group, an optionally substituted cycloalkyl group, an optionally substituted hetero atom containing cycloalkyl group or an optionally substituted monovalent heterocyclic residue;
Y¹, Y² and Y³, which are the same or different with each other, represent an optionally substituted chained alkylene group; and
R¹, R² and R³, which are the same or different, represent an optionally substituted alkyl group, an optionally substituted aralkyl group, or a hydrogen atom.

2. The electrophotographic photoreceptor according to claim 1, wherein the triamine compound is represented by General formula (2): 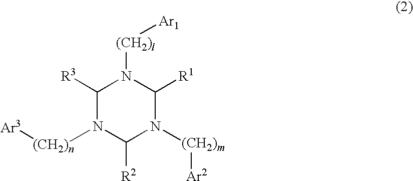
wherein Ar¹, Ar², Ar³, R¹, R² and R³ are synonymous as defined in the General formula (1); n, m and l, which are the same or different with each other, represent an integer of 1 to 3.

3. The electrophotographic photoreceptor according to claim 1, wherein the triamine compound is represented by General formula (3): 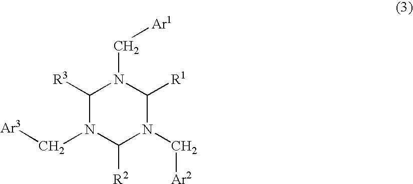
wherein Ar¹, Ar², Ar³, R¹, R² and R³ are synonymous as defined in the General formula (1).

4. The electrophotographic photoreceptor according to claim 1, wherein a ratio A/B between a weight of the charge transporting material A, and a weight of the triamine compound B is 100/0.1 or more and 100/20 or less.

5. The electrophotographic photoreceptor according to claim 1, further comprising an intermediate layer between the conductive supporting members and the monolayer type photoreceptor or the laminate type photoreceptor.

6. An image forming apparatus comprising:
the electrophotographic photoreceptor according to claim 1;
a charging means that charges the electrophotographic photoreceptor;
a light exposing means that exposes the charged electrophotographic photoreceptor to light; and
a developing means that develops an electrostatic latent image formed by the light exposure.

7. The image forming apparatus according to claim 6, wherein the charging means comprises a contact charging.

8. A triamine compound represented by the General formula (1): 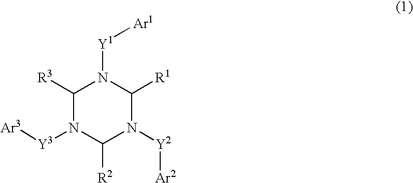
wherein, Ar¹, Ar² and Ar³, which are the same or different with each other, represent an optionally substituted aryl group, an optionally substituted cycloalkyl group, an optionally substituted hetero atom containing cycloalkyl group or an optionally substituted monovalent heterocyclic residue;
Y¹, Y² and Y³, which are the same or different with each other, represent an optionally substituted chained alkylene group, and
R¹, R² and R³, which are the same or different, represent an optionally substituted alkyl group, an optionally substituted aralkyl group, or a hydrogen atom.

9. The triamine compound according to claim 8, wherein
the Ar¹, Ar² and Ar³, which are the same or different with each other, represent a group selected from the group consisting of a phenyl group, a p-methoxyphenyl group, a p-methylphenyl group, a 2-naphthyl group and a cyclohexyl group,
the Y¹, Y² and Y³, which are the same or different with each other, represent a group selected from the group consisting of a methylene group, an ethylene group, a propylene group and a 2,2-dimethyltrimethylene group, and
the R¹, R² and R³, which are the same or different, represent a group selected from the group consisting of a hydrogen atom, a methyl atom, an ethyl atom, a trifluoromethyl group, a benzyl group and a p-methoxybenzyl group.

10. The triamine compound according to claim 8, represented by Formula (4):

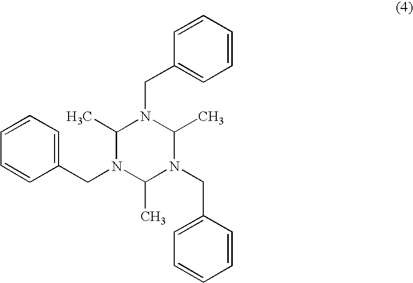

11. The triamine compound according to claim 8, represented by Formula (5);

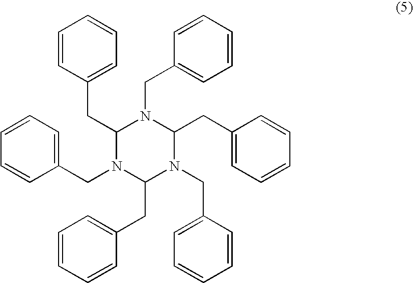

12. A method for producing a triamine compound, wherein the compound of Formula (4) or Formula (5) is obtained by causing an amine compound represented by General formula (6); 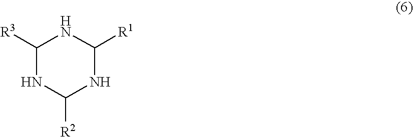
wherein, R¹, R² and R³ represent a methyl group or a benzyl group and a chloro compound represented by Formula (7): 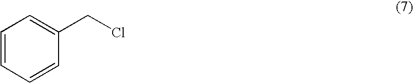
to react in the presence of an organic amine base.
